(19) United States
(12) Patent Application Publication
(10) Pub. No.: US 2008/0193531 A1
(43) Pub. Date: Aug. 14, 2008

(54) COMPOSITIONS FOR IMPROVING GASTROINTESTINAL NUTRIENT AND DRUG ABSORPTION

(75) Inventors: Marc S. Hermelin, Glendale, MO (US); Jonathan David Bortz, Saint Louis, MO (US); R. Saul Levinson, Chesterfield, MO (US)

Correspondence Address: KV PHARMACEUTICAL COMPANY, 4080B WEDGEWAY COURT, EARTH CITY, MO, 63045, US

(73) Assignee: DRUGTECH CORPORATION, Wilmington, DE (US)

(21) Appl. No.: 12/027,538

(22) Filed: Feb. 7, 2008

Related U.S. Application Data
(60) Provisional application No. 60/889,047, filed on Feb. 9, 2007.

(51) Int. Cl. A61K 9/28; A61K 33/06; A61K 33/26; A61K 31/56

(52) U.S. Cl. 424/474; 424/682; 424/646; 514/171

(57) ABSTRACT

The present invention provides pharmaceutical compositions and methods for improving the absorption of nutrients and/or drugs in the gastrointestinal tract of a subject. Typically, the pharmaceutical compositions comprise a first agent that increases the pH of the stomach, and one or more agents selected from a pH lowering agent, a vitamin, a mineral, and a drug.

### This application claims the benefit of U.S. provisional patent application Ser. No. 60/889,047, filed on Feb. 9, 2007, the entire disclosure of which is incorporated by reference herein.

### FIELD OF THE INVENTION

The present invention generally relates to compositions and methods for improving the absorption of nutrients and/or drugs in the gastrointestinal tract of a subject. In particular, the compositions comprise a first agent that increases the pH of the stomach, and one or more agents selected from a pH lowering agent, a vitamin, a mineral, and a drug.

### BACKGROUND OF THE INVENTION

Gastroesophogeal reflux disease (GERD) is characterized by symptoms and/or tissue damage that result from repeated or prolonged exposure of the lining of the esophagus to acidic contents from the stomach. If untreated, GERD can lead to serious health consequences, including stricture formation, esophageal ulcers, or esophageal cancer. Two types of agents are frequently prescribed for the treatment of GERD: H2 blockers and proton pump inhibitors. H2 blockers prevent interactions between the gastric parietal cells that produce acid and histamine, an agent known to stimulate acid secretion. These drugs have a relatively rapid onset of action but a short duration of effectiveness (typically 8-12 hours). Unfortunately, many patients with more severe forms of GERD do not get adequate relief from these H2 blockers.

Proton pump inhibitors (PPIs) are typically prescribed for GERD patients who are not effectively treated with H2 blockers. PPIs are substituted benzimidazoles and are generally administered as enteric-coated tablets or capsules that pass through the stomach intact and are absorbed in the proximal small bowel. Once absorbed, all PPIs have a relatively short plasma half-life but a long duration of action because of their unique mechanism of action. PPIs are lipophilic weak bases that cross the parietal cell membrane and enter the acidic parietal cell canaliculus. In this acidic environment, the PPI becomes protonated, producing the activated sulphenamide form of the drug that binds covalently with the H+/K+ ATPase enzyme, resulting in irreversible inhibition of acid secretion by the proton pump. The parietal cell must then produce new proton pumps or activates resting pumps to resume its acid secretion. Because of the long duration of action of PPIs, they need only to be taken once a day.

Because the gastric pH (which is typically below 2) is raised by PPIs to between 3.5 to 5, and is maintained above 4 for 60% to 70% of the time, the absorption of several nutrients, minerals, vitamins and drugs are negatively affected, which may lead to a variety of nutritional deficiencies and untoward side effects or efficacy issues with prescription medications.

It is well known that the absorption of iron salts is very tightly coupled to the ambient pH of intestinal fluid. While inorganic iron can be absorbed through the entire length of the small intestine, the salts are only absorbed in the proximal duodenum because that is the segment of bowel in which the pH is less than 3, which is necessary to keep the reduced form of iron in solution for absorption. Once the pH exceeds 3, even the more soluble ferrous form of iron precipitates and is not absorbable. Hence, patients on long term treatment with H2 blockers and PPIs generally have gastric pHs well above the levels required for efficient absorption of inorganic iron salts, and hence, iron deficiency is a well recognized complication of these treatment regimens.

The absorption of calcium carbonate in the presence of PPIs has also been described in the literature (O'Connell el al. Am J Med. 2005; 118:778-781), and another publication revealed a significant increase in the risk of hip fractures among patients taking PPIs for at least 1 year (Yang et al. JAMA 2006; 296(24):2947-2953). The authors speculated that calcium malabsorption secondary to acid suppressive therapy could potentially explain the positive association. PPIs may also inhibit the absorption of drugs such as griseofulvin, ketoconazole, itraconazole, iron salts, vitamin B12, cefpodoxime, and enoxacin, many of which are weak bases and require acid for absorption. There is a need, therefore, for formulations comprising a PPI and a supplemental agent, such as a vitamin, a mineral, or a drug, whereby the release of the different agents is optimized so as to enhance their absorption.

### SUMMARY OF THE INVENTION

One aspect of the invention provides a pharmaceutical composition comprising a first agent that increases the pH of the stomach, a second agent that is a pH lowering agent, and at least one of a third agent selected from the group consisting of a vitamin, mineral, and drug.

Yet another aspect of the invention encompasses a multi-layered pharmaceutical composition comprising at least one layer having a first agent that increases the pH of the stomach, and at least one layer having at least one of a second agent selected from a mineral, and a vitamin. The first agent and the second agent may be enteric coated.

Another aspect of the invention provides a pharmaceutical composition comprising a first agent that increases the pH of the stomach, and a second agent that is a pH-lowering agent. Typically, the second agent is enteric coated and released in the small intestine or large intestine.

An additional aspect of the invention provides a pharmaceutical composition comprising a first agent that increases the pH of the stomach, and a drug selected from the group consisting of acid/alkaline-labile drugs, pH dependent drugs, and drugs that are weak acids or weak bases.

Yet a further aspect of the invention encompasses a method for improving the absorption of at least one first agent selected from the group consisting of a nutrient, a vitamin, a mineral, and a drug in a subject. The method involves co-administering to the subject either in combination or as a separate dosage form the first agent and a second agent that is a pH-lowering agent.

An additional aspect of the invention provides a method for improving the absorption of calcium in a subject. The method generally comprises co-administering to the subject calcium and an organic acid.

Another aspect of the invention encompasses a method for improving the absorption of iron in a subject. Typically, the method comprises co-administering to the subject iron and an organic acid.

Other iterations of the invention are described in more detail herein.

### DETAILED DESCRIPTION OF THE INVENTION

The present invention generally provides pharmaceutical compositions formulated in a manner to improve the absorption of various nutrients and/or drugs. In particular, the pharmaceutical compositions provide improved absorption for nutrients and/or drugs that suffer from malabsorption when the gastrointestinal pH, such as the small intestine, is above approximately 4. Advantageously, the pharmaceutical compositions of the invention provide a means to maintain the antacid effect of proton pump inhibitors in the gastric and duodenal mucosa of a subject, while at the same time lowering the pH of the small intestine or the immediate environment (microenvironment) of the active vitamin, mineral or drug to optimize absorption of the vitamin, mineral, or drug.

### (I) Pharmaceutical Compositions

One aspect of the invention provides pharmaceutical compositions comprising at least one agent that increases gastric pH in combination with at least one agent selected from an agent that lowers gastrointestinal pH, vitamin, mineral, drug, buffering agent, and excipients. In one embodiment, the pharmaceutical composition comprises an agent that increases gastric pH, an agent that lowers gastrointestinal pH, and a mineral. In an alternative of this embodiment, the pharmaceutical composition comprises an agent that increases gastric pH, an agent that lowers gastrointestinal pH, and a vitamin. In yet another alternative embodiment, the pharmaceutical composition comprises an agent that increases gastric pH, an agent that lowers gastrointestinal pH, and a drug. In another embodiment, the pharmaceutical composition comprises an agent that increases gastric pH and a vitamin. In an additional embodiment, the pharmaceutical composition comprises an agent that increases gastric pH and a mineral. In yet another embodiment, the pharmaceutical composition comprises an agent that increases gastric pH and a drug. In still another embodiment, the pharmaceutical composition comprises an agent that increases gastric pH and an agent that lowers gastrointestinal pH. Suitable agents for lowering gastric pH, for increasing gastrointestinal pH, minerals, vitamins, drugs, buffering agents, and excipients are described in more detail below.

(a) Agents that Increase Gastric pH

Generally speaking, suitable agents that increase gastric pH include agents that increase the pH of gastric acid in the stomach lumen from physiological level of about 2 to a pH greater than about 3 and more typically, greater than about 4. The agent may sustain the elevated pH levels for approximately 5% to 10%, 10% to 15%, 15% to 20%, 20% to 25%, 25% to 30%, 30% to 35%, 35% to 40%, 40% to 45%, 45% to 50%, 50% to 55%, 55% to 60%, 60% to 65%, 65% to 70%, 70% to 75%, 75% to 80%, 80% to 85%, 85% to 90%, 90% to 95%, or greater than about 95% of the time on a daily basis. A skilled artisan using methods generally known in the art can readily measure the pH of gastric acid in the stomach lumen.

One suitable class of agents that increase gastric pH includes proton pump inhibitors. Proton pump inhibitors are typically acid labile pharmaceutical agents that substantially inhibit H⁺/K⁺ATPase. In one embodiment, the proton pump inhibitor can be a substituted bicyclic aryl-imidazole, wherein the aryl group can be, e.g., a pyridine, a phenyl, or a pyrimidine group and is attached to the 4- and 5-positions of the imidazole ring. Proton pump inhibitors comprising a substituted bicyclic aryl-imidazoles include, but are not limited to, omeprazole, hydroxyomeprazole, esomeprazole, lansoprazole, pantoprazole, rabeprazole, dontoprazole, habeprazole, perprazole, tenatoprazole, ransoprazole, pariprazole, leminoprazole. Other proton pump inhibitors include but are not limited to: soraprazan (Altana); ilaprazole (U.S. Pat. No. 5,703,097) (II-Yang); AZD-0865 (AstraZeneca); YH-1885 (PCT Publication WO 96/05177) (SB-641257) (2-pyrimidinamine, 4-(3,4-dihydro-1-methyl-2(1H)-isoquinolinyl)-N-(4-fluorophenyl)-5,6-dimet-hyl-monohydrochloride)(YuHan); BY-112 (Altana); SPI-447 (Imidazo(1,2-a)thieno(3,2-c)pyridin-3-amine,5-methyl-2-(2-methyl-3-thieny-I) (Shinnippon); 3-hydroxymethyl-2-methyl-9-phenyl-7H-8,9-dihydro-pyrano(2,3-c)-imidazo(1,-2-a)pyridine (PCT Publication WO 95/27714) (AstraZeneca); Pharmaprojects No. 4950 (3-hydroxymethyl-2-methyl-9-phenyl-7H-8,9-dihydro-pyrano(2,3-c)-imidazo(1,2-a)pyridine) (AstraZeneca, ceased) WO 95/27714; Pharmaprojects No. 4891 (EP 700899) (Aventis); Pharmaprojects No. 4697 (PCT Publication WO 95/32959) (AstraZeneca); H-335/25 (AstraZeneca); T-330 (Saitama 335) (Pharmacological Research Lab); Pharmaprojects No. 3177 (Roche); BY-574 (Altana); Pharmaprojects No. 2870 (Pfizer); AU-1421 (EP 264883) (Merck); AU-2064 (Merck); AY-28200 (Wyeth); Pharmaprojects No. 2126 (Aventis); WY-26769 (Wyeth); pumaprazole (PCT Publication WO 96/05199) (Altana); YH-1238 (YuHan); Pharmaprojects No. 5648 (PCT Publication WO 97/32854) (Dainippon); BY-686 (Altana); YM-020 (Yamanouchi); GYKI-34655 (Ivax); FPL-65372 (Aventis); Pharmaprojects No. 3264 (EP 509974) (AstraZeneca); nepaprazole (To a Eiyo); HN-11203 (Nycomed Pharma); OPC-22575; pumilacidin A (BMS); saviprazole (EP 234485) (Aventis); SK and F-95601 (GSK, discontinued); Pharmaprojects No. 2522 (EP 204215) (Pfizer); S-3337 (Aventis); RS-13232A (Roche); AU-1363 (Merck); SK and F-96067 (EP 259174) (Altana); SUN 8176 (Daiichi Phama); Ro-18-5362 (Roche); ufiprazole (EP 74341) (AstraZeneca); and Bay-p-1455 (Bayer). Additional proton pump inhibitors suitable for use include, without limitation, those described in the following U.S. Pat. Nos. 4,628,098; 4,689,333; 4,786,505; 4,853,230; 4,965,269; 5,021,433; 5,026,560; 5,045,321; 5,093,132; 5,430,042; 5,433,959; 5,576,025; 5,639,478; 5,703,110; 5,705,517; 5,708,017; 5,731,006; 5,824,339; 5,855,914; 5,879,708; 5,948,773; 6,017,560; 6,123,962; 6,187,340; 6,296,875; 6,319,904; 6,328,994; 4,255,431; 4,508,905; 4,636,499; 4,738,974; 5,690,960; 5,714,504; 5,753,265; 5,817,338; 6,093,734; 6,013,281; 6,136,344; 6,183,776; 6,328,994; 6,479,075; 6,559,167, each of which is hereby incorporated by reference in their entirety.

Pharmaceutical compositions of the invention may include proton pump inhibitors in an amount ranging from about 1 mg to about 500 mg, from about 1 mg to about 200 mg, or from about 5 mg to about 100 mg per dosage. Examples of preferred dosages for particular proton pump inhibitors are: about 5 mg to about 50 mg omeprazole; about 5 mg to about 100 mg esomeprazole; about 15 mg to about 150 mg lansoprazole; about 10 mg to about 200 mg pantoprazole; and about 5 mg to about 100 mg rabeprazole.

In another embodiment, the agent that increases gastric pH is a histamine H2-receptor antagonist, commonly known as an H2 blocker. H2-blockers generally inhibit secretion of acid by the parietal cells in the stomach lining, and thereby, cause gastric acid pH to increase. Suitable H2 blockers include cimetidine (commercially available as Tagamet or Tagamet HB); ranitidine (commercially available as Zantac); famotidine (commercially available as Pepcid AC or Pepcid); ebrotidine; pabutidine; lafutidine; and nizatidine (commercially available as Axid AR or Axid). Generally speaking, the pharmaceutical composition may include an amount of an H2 blocker ranging from about 1 mg to about 300 mg, from about 5 mg to about 150 mg, or from about 10 mg to about 100 mg.

(b) Agents that Lower Gastrointestinal pH

The pharmaceutical composition may comprise an agent that decreases gastrointestinal pH. Typically, the agent will be formulated such that it is released within the gastrointestinal tract at approximately the same location and time as a nutrient or drug that is poorly absorbed at pH levels greater than about 2 or 3. It is believed, without being bound to any particular theory, that co-administration of the pH lowering agent and the aforementioned nutrient and/or drug will generally improve the absorption levels of the nutrient or drug. The pH lowering agent may decrease the pH of the bulk fluid of the gastrointestinal tract, as well as lower the pH of a microenvironment at the gastrointestinal mucosa. As will be appreciated by a skilled artisan the extent of increased absorption of the nutrient and/or drug can and will vary depending upon the pH, the choice of pH lowering agents, nutrients, and drugs, and their respective pharmaceutical formulation. By way of non-limiting example, absorption may be increased from about 1% to about 5%, about 5% to about 10%, about 10% to about 15%, about 15% to about 20%, about 20% to about 25%, about 25% to about 30%, about 30% to about 35%, about 35% to about 40%, about 40% to about 45%, about 45% to about 50%, about 50% to about 55%, about 55% to about 60%, about 60% to about 65%, about 65% to about 70%, about 70% to about 75%, about 75% to about 80%, about 80% to about 85%, about 85% to about 90%, about 90% to about 95%, or greater than about 95% compared to administration of the nutrient or drug by itself (i.e., without the pH lowering agent). The amount of nutrient or drug absorption can be reliably measured using methods generally known in the art.

Suitable pH lowering agents include organic acids selected from the aliphatic, cycloaliphatic, aromatic, heterocyclic, carboxylic and sulfonic classes of organic acids. The organic acid may be selected from small monocarboxylic, dicarboxylic or tricarboxylic acids, or any active derivative or salt thereof. Non-limiting examples of suitable organic acids include acetic, acetylglutamic, acetylsalicylic, adipic, anthranilic, ascorbic, aspartic, azelaic, benzoic, cinnamic, citric, embonic (pamoic), formic, fumaric, gluconic, glucuronic, glutamic, glutaric, glyceric, glycolic, glycocolic, glyoxylic, p-hydroxybenzoic, isocitric, isovaleric, lactic, maleic, malic, malonic, mandelic, mesylic, oxalic, oxaloacetic, oxalosuccinic, palmitic, phenylacetic, phosphoglyceric, pimelic, propionic, pyruvic, salicylic, sebasic, suberic, succinic, stearic, tartaric, valeric, methanesulfonic, ethanesulfonic, benzenesulfonic, pantothenic, toluenesulfonic, 2-hydroxyethanesulfonic, sulfanilic, cyclohexylaminosulfonic, algenic, β-hydroxybutyric, galactaric, and galacturonic acid. Preferred organic acids include acetic acid, aspartic acid, citric acid, fumaric acid, lactic acid, malic acid, pyruvic acid, and tartaric acid, more preferred organic acids include ascorbic acid and glutamic acid, and the most preferred organic acid is succinic acid.

Generally speaking, the pharmaceutical composition may include an amount of organic acid necessary to achieve a pharmacological effect of lowering the gastrointestinal tract to a desired pH without producing undue adverse side effects in the subject. In this context, the amount of organic acid may be quantified as the amount needed to reduce the pH of the gastrointestinal tract to a pH less than about 4, about 3.75, about 3.5, about 3.25, about 3.0, about 2.75, about 2.5, about 2.25, or less than about 2.0. By way of non-limiting example, the amount of organic acid in any particular pharmaceutical formulation may range from about 1 mg to about 25,000 mg, from about 5 mg to about 1000 mg, from about 100 mg to about 750 mg, or from about 150 mg to about 500 mg per dosage. For pediatric formulations, the amount of organic acid may be as low as 0.50 mg of organic acid per kilogram of body weight per dosage. The pH lowering agent may also not be measurable in the gastrointestinal fluid, but may be present only in the microenvironment of the active vitamin, mineral or drug, and yet may exert an effect that could render that vitamin, mineral or drug to be more easily absorbed. This, for example, is one of the recognized mechanisms by which ascorbic acid (vitamin C) is known to promote the absorption of ferrous salts.

(c) Minerals

The pharmaceutical composition may include one or more minerals or mineral sources. Non-limiting examples of minerals include, without limitation, calcium, iron, chromium, copper, iodine, zinc, magnesium, manganese, molybdenum, phosphorus, potassium, and selenium. Suitable forms of any of the foregoing minerals include soluble mineral salts, slightly soluble mineral salts, insoluble mineral salts, chelated minerals, mineral complexes, non-reactive minerals such as carbonyl minerals, and reduced minerals, and combinations thereof.

Suitable forms of zinc, include, zinc chelates (complexes of zinc and amino acids, dipeptides, or polypeptides), zinc acetate, zinc aspartate, zinc citrate, zinc glucoheptonate, zinc gluconate, zinc glycerate, zinc picolinate, zinc monomethionine and zinc sulfate.

Examples of suitable forms of copper include copper chelates, cupric oxide, copper gluconate, copper sulfate, and copper amino acid chelates

Suitable forms of calcium include calcium alpha-ketoglutarate, calcium acetate, calcium alginate, calcium ascorbate, calcium aspartate, calcium caprylate, calcium carbonate, calcium chelates, calcium chloride, calcium citrate, calcium citrate malate, calcium formate, calcium glubionate, calcium glucoheptonate, calcium gluconate, calcium glutarate, calcium glycerophosphate, calcium lactate, calcium lysinate, calcium malate, calcium orotate, calcium oxalate, calcium oxide, calcium pantothenate, calcium phosphate, calcium pyrophosphate, calcium succinate, calcium sulfate, calcium undecylenate, coral calcium, dicalcium citrate, dicalcium malate, dihydroxycalcium malate, dicalcium phosphate, and tricalcium phosphate.

In an exemplary formulation, the pharmaceutical composition generally will include iron. A variety of suitable forms of iron may be included in the pharmaceutical composition of the invention. In one embodiment, the iron may be in the form of chelates, such as Ferrochel™ (Albion International, Inc., Clearfield, Utah) a commercially available bis-glycine chelate of iron, and Sumalate™ (Albion International, Inc., Clearfield, Utah) a commercially available ferrous asparto glycinate. For example, amino acid chelates are becoming well accepted as a means of increasing the metal content in biological tissues of subjects. Amino acid chelates are products resulting from the reaction of a polypeptide, dipeptide or naturally occurring alpha amino acid with a metal ion having a valence of two or more. The alpha amino acid and metal ion form a ring structure wherein the positive electrical charges of the metal ion are neutralized by the electrons of the carboxylate or free amino groups of the alpha amino acid. Although the term amino acid as used herein refers only to products obtainable through protein hydrolysis, synthetically produced amino acids are not to be excluded provided they are the same as those obtained through protein hydrolysis. Accordingly, protein hydrolysates such as polypeptides, dipeptides and naturally occurring alpha amino acids are collectively referred to as amino acids. Additional suitable amino acid chelates include for example but are not limited to ethylenediaminetetraacetic acid (EDTA), monohydroxyethylethylenediaminetriacetic acid, diethylenetriaminepentaacetic acid, monohydroxyethyldiglycine and dihydroxyethylglycine.

Other suitable forms of iron for purposes of the present invention include for example but are not limited to soluble iron salts, slightly soluble iron salts, insoluble iron salts, chelated iron, iron complexes, non-reactive iron such as carbonyl iron and reduced iron, and combinations thereof.

Suitable chelated iron complexes are disclosed in U.S. Pat. Nos. 4,599,152 and 4,830,716, each incorporated herein by reference.

Examples of suitable soluble iron salts include but are not limited to ferric hypophosphite, ferric albuminate, ferric chloride, ferric citrate, ferric oxide saccharate, ferric ammonium citrate, ferrous chloride, ferrous gluconate, ferrous iodide, ferrous sulfate, ferrous lactate, ferrous fumarate, heme, ferric trisglycinate, ferrous bisglycinate, ferric nitrate, ferrous hydroxide saccharate, ferric sulfate, ferric gluconate, ferric aspartate, ferrous sulfate heptahydrate, ferrous phosphate, ferric ascorbate, ferrous formate, ferrous acetate, ferrous malate, ferrous glutamate, ferrous cholinisocitrate, ferroglycine sulfate, ferric oxide hydrate, ferric pyrophosphate soluble, ferric hydroxide saccharate, ferric manganese saccharate, ferric subsulfate, ferric ammonium sulfate, ferrous ammonium sulfate, ferric sesquichloride, ferric choline citrate, ferric manganese citrate, ferric quinine citrate, ferric sodium citrate, ferric sodium edetate, ferric formate, ferric ammonium oxalate, ferric potassium oxalate, ferric sodium oxalate, ferric peptonate, ferric manganese peptonate, other pharmaceutically acceptable iron salts, and combinations thereof.

Examples of suitable slightly soluble iron salts include but are not limited to ferric acetate, ferric fluoride, ferric phosphate, ferric pyrophosphate, ferrous pyrophosphate, ferrous carbonate saccharated, ferrous carbonate mass, ferrous succinate, ferrous citrate, ferrous tartrate, ferric fumarate, ferric succinate, ferrous hydroxide, ferrous nitrate, ferrous carbonate, ferric sodium pyrophosphate, ferric tartrate, ferric potassium tartrate, ferric subcarbonate, ferric glycerophosphate, ferric saccharate, ferric hydroxide saccharate, ferric manganese saccharate, ferrous ammonium sulfate, other pharmaceutically acceptable iron salts, and combinations thereof.

Suitable examples of insoluble iron salts include but are not limited to ferric sodium pyrophosphate, ferrous carbonate, ferric hydroxide, ferrous oxide, ferric oxyhydroxide, ferrous oxalate, other pharmaceutically acceptable iron salts and combinations thereof.

Examples of suitable iron complexes include but are not limited to polysaccharide-iron complex, methylidine-iron complex, ethylenediaminetetraacetic acid (EDTA)-iron complex, phenanthrolene iron complex, p-toluidine iron complex, ferrous saccharate complex, ferrlecit, ferrous gluconate complex, ferrum vitis, ferrous hydroxide saccharate complex, iron-arene sandwich complexes, acetylacetone iron complex salt, iron-dextran complex, iron-dextrin complex, iron-sorbitol-citric acid complex, saccharated iron oxide, ferrous fumarate complex, iron porphyrin complex, iron phtalocyamine complex, iron cyclam complex, dithiocarboxy-iron complex, desferrioxamine-iron complex, bleomycin-iron complex, ferrozine-iron complex, iron perhaloporphyrin complex, alkylenediamine-N,N-disuccinic acid iron(III) complex, hydroxypyridone-iron(III) complex, aminoglycoside-iron complex, transferrin-iron complex, iron thiocyanate complex, iron complex cyanides, porphyrinato iron(III) complex, polyaminopolycarbonate iron complexes, dithiocarbamate iron complex, adriamycin iron complex, anthracycline-iron complex, N-methyl-D-glucamine dithiocarbamate (MGD)-iron complex, ferrioxamine B, ferrous citrate complex, ferrous sulfate complex, ferric gluconate complex, ferrous succinate complex, polyglucopyranosyl iron complex, polyaminodisuccinic acid iron complex, biliverdin-iron complex, deferiprone iron complex, ferric oxyhydride-dextran complex, dinitrosyl dithiolato iron complex, iron lactoferrin complexes, 1,3-ethylenediaminetetraacetic acid (EDTA) ferric complex salts, diethylenetriaminepentaacetic acid iron complex salts, cyclohexanediaminetetraacetic acid iron complex salts, methyliminodiacetic acid iron complex salts, glycol ether diaminetetraacetic acid iron complex salts, ferric hydroxypyrone complexes, ferric succinate complex, ferric chloride complex, ferric glycine sulfate complex, ferric aspartate complex, sodium ferrous gluconate complex, ferrous hydroxide polymaltose complex, other pharmaceutically acceptable iron complexes and combinations thereof.

Suitable forms of iron for purposes of the present invention also include iron compounds designated as “slow dissolving” or “slow acting” and iron compounds designated as “fast dissolving” or “fast acting”. Compositions of the present invention may optionally include at least two iron compounds, e.g., at least one iron compound designated slow acting and at least one iron compound designated as fast acting. The use of two such differing iron compounds in a formulation is disclosed in U.S. Pat. No. 6,521,247, incorporated herein in its entirety by reference. Compositions of the present invention may also include extended release iron compounds and/or controlled release iron compounds.

Generally speaking, the pharmaceutical composition may include one or more forms of an effective amount of any of the minerals described herein or otherwise known in the art. Exemplary minerals include calcium, iron, and zinc. An “effective amount” of a mineral typically quantifies an amount at least about 10% of the United States Recommended Daily Allowance (“RDA”) of that particular mineral for a subject. It is contemplated, however, that amounts of certain minerals exceeding the RDA may be beneficial for certain subjects. For example, the amount of a given mineral may exceed the applicable RDA by 100%, 200%, 300%, 400% or 500% or more. Typically, the amount of mineral included in the pharmaceutical composition may range from about 1 mg to about 1500 mg, about 5 mg to about 500 mg, or from about 150 mg to about 500 mg per dosage.

(d) vitamins

Suitable vitamins for use in the pharmaceutical compositions include vitamin C, vitamin A, vitamin E, vitamin B12, vitamin K, riboflavin, niacin, vitamin D, vitamin B6, folic acid, pyridoxine, thiamine, pantothenic acid, and biotin. The form of the vitamin may include salts of the vitamin, derivatives of the vitamin, compounds having the same or similar activity of a vitamin, and metabolites of a vitamin. By way of non-limiting example, the pharmaceutical composition may include ascorbic acid (i.e., vitamin C), salts of ascorbic acid, derivatives of ascorbic acid, compounds having Vitamin C activity, carbohydrates such as but not limited to mannitol, sorbitol, xylose, inositol, fructose, sucrose, lactose, and glucose, calcium, copper, sodium molybdate, amino acids and combinations thereof. “Compounds having Vitamin C activity” means Vitamin C (L-ascorbic acid) and any derivative thereof that exhibits ascorbic activity as determined by the standard iodine titration test. Derivatives of ascorbic acid include, for example, oxidation products such as dehydroascorbic acid and edible salts of ascorbic acid such as for example but not limited to calcium ascorbate, sodium ascorbate, magnesium ascorbate, potassium ascorbate and zinc ascorbate. Metabolites of ascorbic acid and its derivatives include for example but are not limited to aldo-lactones and edible salts of aldonic acids. Compositions of the present invention preferably include one or more ascorbic acid metabolites, namely, L-threonic acid, L-xylonic acid and L-lyxonic acid. A preferred form of ascorbic acid for purposes of the present invention is Ester C® (Zila Nutraceuticals, Inc., Prescott, Arizona), as disclosed in U.S. Pat. Nos. 4,822,816 and 5,070,085, each incorporated herein by reference.

The pharmaceutical composition may include one or more forms of an effective amount of any of the vitamins described herein or otherwise known in the art. Exemplary vitamins include vitamin B12, vitamin C, vitamin D, and vitamin E. An “effective amount” of a vitamin typically quantifies an amount at least about 10% of the United States Recommended Daily Allowance (“RDA”) of that particular vitamin for a subject. It is contemplated, however, that amounts of certain vitamins exceeding the RDA may be beneficial for certain subjects. For example, the amount of a given vitamin may exceed the applicable RDA by 100%, 200%, 300%, 400% or 500% or more.

(e) Drugs

The pharmaceutical composition may include a drug. In some embodiments, the drug may be an acid/alkaline-labile drug, a pH dependent drug, or a drug that is a weak acid or a weak base. Examples of acid-labile drugs include statins (e.g., pravastatin, fluvastatin and atorvastatin), antiobiotics (e.g., penicillin G, ampicillin, streptomycin, erythromycin, clarithromycin and azithromycin), nucleoside analogs [e.g., dideoxyinosine (ddl or didanosine), dideoxyadenosine (ddA), dideoxycytosine (ddC)], salicylates (e.g, aspirin), digoxin, bupropion, pancreatin, midazolam, and methadone. Drugs that are only soluble at acid pH include nifedipine, emonapride, nicardipine, amosulalol, noscapine, propafenone, quinine, dipyridamole, josamycin, dilevalol, labetalol, enisoprost, and metronidazole. Drugs that are weak acids include phenobarbital, phenytoin, zidovudine (AZT), salicylates (e.g., aspirin), propionic acid compounds (e.g., ibuprofen), indole derivatives (e.g., indomethacin), fenamate compounds (e.g., meclofenamic acid), pyrrolealkanoic acid compounds (e.g., tolmetin), cephalosporins (e.g., cephalothin, cephalaxin, cefazolin, cephradine, cephapirin, cefamandole, and cefoxitin), 6-fluoroquinolones, and prostaglandins. Drugs that are weak bases include adrenergic agents (e.g., ephedrine, desoxyephedrine, phenylephrine, epinephrine, salbutamol, and terbutaline), cholinergic agents (e.g., physostigmine and neostigmine), antispasmodic agents (e.g., atropine, methantheline, and papaverine), curariform agents (e.g., chlorisondamine), tranquilizers and muscle relaxants (e.g., fluphenazine, thioridazine, trifluoperazine, chlorpromazine, and triflupromazine), antidepressants (e.g., amitriptyline and nortriptyline), antihistamines (e.g., diphenhydramine, chlorpheniramine, dimenhydrinate, tripelennamine, perphenazine, chlorprophenazine, and chlorprophenpyridamine), cardioactive agents (e.g., verapamil, diltiazem, gallapomil, cinnarizine, propranolol, metoprolol and nadolol), antimalarials (e.g., chloroquine), analgesics (e.g., propoxyphene and meperidine), antifungal agents (e.g., ketoconazole and itraconazole), antimicrobial agents (e.g., cefpodoxime, proxetil, and enoxacin), caffeine, theophylline, and morphine.

In another embodiment, the drug may be a biphosphonate or another drug used to treat osteoporosis. Non-limiting examples of a biphosphonate include alendronate, ibandronate, risedronate, zoledronate, pamidronate, neridronate, olpadronate, etidronate, clodronate, and tiludronate. Other suitable drugs include estrogen, selective estrogen receptor modulators (SERMs), and parathyroid hormone (PTH) drugs. In yet another embodiment, the drug may be an antibacterial agent. Suitable antibiotics include aminoglycosides (e.g., amikacin, gentamicin, kanamycin, neomycin, netilmicin streptomycin, and tobramycin), carbecephems (e.g., loracarbef) a carbapenem (e.g., certapenem, imipenem, and meropenem) cephalosporins (e.g., cefadroxil cefazolin, cephalexin, cefaclor cefamandole, cephalexin, cefoxitin, cefprozil, cefuoxime, cefiximem cefdinir, cefditoren, cefoperazone, ceftaxime, cefpodoxime, ceftazdime, ceftibuten, ceftizoxime, and ceftriaxone), macrolides (e.g., azithromycin, clarithromycin, dirthromycin, erythrmoycin, and troleandomycin), monobactam, penicillins (e.g., amoxicillin, ampicillin, carbenicillin, cloxacillin, dicloxacillin, nafillin, oxacillin, penicillin G, penicillin V, piperacillin, and ticarcillin), polypeptides (e.g., bacitracin, colistin, and polymyxin B), quinolones (e.g., ciprofloxacin. enoxacin, gatifloxacin, levofloxacin, lomefloxacin, moxifloxacin, norfloxacin, ofloxacin, and trovafloxacin), sulfonamides (e.g., mafenide, sulfacetamide, sulfacethizol e, sulfsalazine, sulfisoxazole, and trimethoprim-sulfmethoxazole), and tetracyclines (e.g., demeclocycline, doxycycline, minocycline, and oxytetracycline). In an alternate embodiment, the drug may be an antiviral protease inhibitor (e.g., amprenavir, fosamprenavir, indinavir, lopinavir/ritonavir, ritonavir, saquinavir, and nelfinavir). In a still another embodiment, the drug may be a cardiovascular drug. Examples of suitable cardiovascular agents include cardiotonic agents (e.g., digitalis (digoxin), ubidecarenone, and dopamine), vasodilating agents (e.g., nitroglycerin, captopril, dihydralazine, diltiazem, and isosorbide dinitrate), antihypertensive agents (e.g., alpha-methyldopa, chlortalidone, reserpine, syrosingopine, rescinnamine, prazosin, phentolamine, felodipine, propanolol, pindolol, labetalol, clonidine, captopril, enalapril, and lisonopril), beta blockers (e.g., levobunolol, pindolol, timolol maleate, bisoprolol, carvedilol, and butoxamine), alpha blockers (e.g., doxazosin, prazosin, phenoxybenzamine, phentolamine, tamsulosin, alfuzosin, and terazosin), calcium channel blockers (e.g., amlodipine, felodipine, nicardipine, nifedipine, nimodipine, nisoldipine, nitrendipine, lacidipine, lercanidipine, verapamil, gallopamil, and diltiazem), and anticlot agents (e.g., dipyrimadole).

(f) Buffering Agents

In certain embodiments, the pharmaceutical composition may include at least one buffering agent. The buffering agent will generally be an antacid. Suitable antacids include those comprised of alkali metal (a Group IA metal including, but not limited to, lithium, sodium, potassium, rubidium, cesium, and francium) or alkaline earth metal (Group IIA metal including, but not limited to, beryllium, magnesium, calcium, strontium, barium, radium) carbonates, phosphates, bicarbonates, citrates, borates, acetates, phthalates, tartrate, succinates and the like, such as sodium or potassium phosphate, citrate, borate, acetate, bicarbonate and carbonate. Non-limiting examples of suitable antacids include an amino acid, an alkali salt of an amino acid, aluminum hydroxide, aluminum hydroxide/magnesium carbonate/calcium carbonate co-precipitate, aluminum magnesium hydroxide, aluminum hydroxide/magnesium hydroxide co-precipitate, aluminum hydroxide/sodium bicarbonate co-precipitate, aluminum glycinate, calcium acetate, calcium bicarbonate, calcium borate, calcium carbonate, calcium citrate, calcium gluconate, calcium glycerophosphate, calcium hydroxide, calcium lactate, calcium phthalate, calcium phosphate, calcium succinate, calcium tartrate, dibasic sodium phosphate, dicalcium malate, dihydroxycalcium malate, dipotassium hydrogen phosphate, dipotassium phosphate, disodium hydrogen phosphate, disodium succinate, dry aluminum hydroxide gel, L-arginine, magnesium acetate, magnesium aluminate, magnesium borate, magnesium bicarbonate, magnesium carbonate, magnesium citrate, magnesium gluconate, magnesium hydroxide, magnesium lactate, magnesium metasilicate aluminate, magnesium oxide, magnesium phthalate, magnesium phosphate, magnesium silicate, magnesium succinate, magnesium tartrate, potassium acetate, potassium carbonate, potassium bicarbonate, potassium borate, potassium citrate, potassium metaphosphate, potassium phthalate, potassium phosphate, potassium polyphosphate, potassium pyrophosphate, potassium succinate, potassium tartrate, sodium acetate, sodium bicarbonate, sodium borate, sodium carbonate, sodium citrate, sodium gluconate, sodium hydrogen phosphate, sodium hydroxide, sodium lactate, sodium phthalate, sodium phosphate, sodium polyphosphate, sodium pyrophosphate, sodium sesquicarbonate, sodium succinate, sodium tartrate, sodium tripolyphosphate, synthetic hydrotalcite, tetrapotassium pyrophosphate, tetrasodium pyrophosphate, tripotassium phosphate, trisodium phosphate, and trometamol.

The amount of antacid present in the pharmaceutical formulation may generally range from about 200 mg to about 3500 mg per dosage. In other embodiments, the amount of antacid present in the pharmaceutical formulation is about 200 mg, or about 300 mg, or about 400 mg, or about 500 mg, or about 600 mg, or about 700 mg, or about 800 mg, or about 900 mg, or about 1000 mg, or about 1100 mg, or about 1200 mg, or about 1300 mg, or about 1400 mg, or about 1500 mg, or about 1600 mg, or about 1700 mg, or about 1800 mg, or about 1900 mg, or about 2000 mg, or about 2100 mg, or about 2200 mg, or about 2300 mg, or about 2400 mg, or about 2500 mg, or about 2600 mg, or about 2700 mg, or about 2800 mg, or about 2900 mg, or about 3000 mg, or about 3200 mg, or about 3500 mg, or about 10,000 mg, or about 20,000 mg, or about 25,000 mg.

(g) Excipients

A variety of commonly used excipients in pharmaceutical formulations may be selected on the basis of compatibility with the pharmaceutically active agents, and the release profile properties of the desired dosage form, such as release location. Non-limiting examples of suitable excipients include an agent selected from the group consisting of non-effervescent disintegrants, a coloring agent, a flavor-modifying agent, an oral dispersing agent, a stabilizer, a preservative, a diluent, a compaction agent, a lubricant, a filler, a binder, taste masking agents, an effervescent disintegration agent, and combinations of any of these agent.

In one embodiment, the excipient is a binder. Suitable binders include starches, pregelatinized starches, gelatin, polyvinylpyrolidone, cellulose, methylcellulose, sodium carboxymethylcellulose, ethylcellulose, polyacrylamides, polyvinyloxoazolidone, polyvinylalcohols, C₁₂-C₁₈ fatty acid alcohol, polyethylene glycol, polyols, saccharides, oligosaccharides, polypeptides, oligopeptides, and combinations thereof. The polypeptide may be any arrangement of amino acids ranging from about 100 to about 300,000 daltons.

In another embodiment, the excipient may be a filler. Suitable fillers include carbohydrates, inorganic compounds, and polyvinilpirrolydone. By way of non-limiting example, the filler may be calcium sulfate, both di- and tri-basic, starch, calcium carbonate, magnesium carbonate, microcrystalline cellulose, dibasic calcium phosphate, magnesium carbonate, magnesium oxide, calcium silicate, talc, modified starches, lactose, sucrose, mannitol, and sorbitol.

The excipient may comprise a non-effervescent disintegrant. Suitable examples of non-effervescent disintegrants include starches such as corn starch, potato starch, pregelatinized and modified starches thereof, sweeteners, clays, such as bentonite, micro-crystalline cellulose, alginates, sodium starch glycolate, gums such as agar, guar, locust bean, karaya, pecitin, and tragacanth.

In another embodiment, the excipient may be an effervescent disintegrant. By way of non-limiting example, suitable effervescent disintegrants include sodium bicarbonate in combination with citric acid and sodium bicarbonate in combination with tartaric acid.

The excipient may comprise a preservative. Suitable examples of preservatives include antioxidants, such as a-tocopherol or ascorbate, and antimicrobials, such as parabens, chlorobutanol or phenol.

In another embodiment, the excipient may include a diluent. Diluents suitable for use include pharmaceutically acceptable saccharide such as sucrose, dextrose, lactose, microcrystalline cellulose, fructose, xylitol, and sorbitol; polyhydric alcohols; a starch; pre-manufactured direct compression diluents; and mixtures of any of the foregoing.

The excipient may include flavors. Flavors incorporated into the outer layer may be chosen from synthetic flavor oils and flavoring aromatics and/or natural oils, extracts from plants, leaves, flowers, fruits, and combinations thereof. By way of example, these may include cinnamon oils, oil of wintergreen, peppermint oils, clover oil, hay oil, anise oil, eucalyptus, vanilla, citrus oil, such as lemon oil, orange oil, grape and grapefruit oil, fruit essences including apple, peach, pear, strawberry, raspberry, cherry, plum, pineapple, and apricot.

In another embodiment, the excipient may include a sweetener. By way of non-limiting example, the sweetener may be selected from glucose (corn syrup), dextrose, invert sugar, fructose, and mixtures thereof (when not used as a carrier); saccharin and its various salts such as the sodium salt; dipeptide sweeteners such as aspartame; dihydrochalcone compounds, glycyrrhizin; Stevia Rebaudiana (Stevioside); chloro derivatives of sucrose such as sucralose; sugar alcohols such as sorbitol, mannitol, sylitol, and the like. Also contemplated are hydrogenated starch hydrolysates and the synthetic sweetener 3,6-dihydro-6-methyl-1,2,3-oxathiazin-4-one-2,2-dioxide, particularly the potassium salt (acesulfame-K), and sodium and calcium salts thereof.

In another embodiment, the excipient may be a lubricant. Suitable non-limiting examples of lubricants include magnesium stearate, calcium stearate, zinc stearate, hydrogenated vegetable oils, sterotex, polyoxyethylene monostearate, talc, polyethyleneglycol, sodium benzoate, sodium lauryl sulfate, magnesium lauryl sulfate, and light mineral oil.

The excipient may be a dispersion enhancer. Suitable dispersants may include starch, alginic acid, polyvinylpyrrolidones, guar gum, kaolin, bentonite, purified wood cellulose, sodium starch glycolate, isoamorphous silicate, and microcrystalline cellulose as high HLB emulsifier surfactants.

Depending upon the embodiment, it may be desirable to provide a coloring agent in the outer layer. Suitable color additives include food, drug and cosmetic colors (FD&C), drug and cosmetic colors (D&C), or external drug and cosmetic colors (Ext. D&C). These colors or dyes, along with their corresponding lakes, and certain natural and derived colorants may be suitable for use in the present invention depending on the embodiment.

The exipient may include a taste-masking agent. Taste-masking materials include, e.g., cellulose hydroxypropyl ethers (HPC) such as Klucel®, Nisswo HPC and PrimaFlo HP22; low-substituted hydroxypropyl ethers (L-HPC); cellulose hydroxypropyl methyl ethers (HPMC) such as Seppifilm-LC, Pharmacoat.R™., Metolose SR, Opadry YS, PrimaFlo, MP3295A, Benecel MP824, and Benecel MP843; methylcellulose polymers such as Methocel® and Metolose®; Ethylcelluloses (EC) and mixtures thereof such as E461, Ethocel.R™., Aqualon®-EC, Surelease; Polyvinyl alcohol (PVA) such as Opadry AMB; hydroxyethylcelluloses such as Natrosol®; carboxymethylcelluloses and salts of carboxymethylcelluloses (CMC) such as Aualon®-CMC; polyvinyl alcohol and polyethylene glycol co-polymers such as Kollicoat IR®; monoglycerides (Myverol), triglycerides (KLX), polyethylene glycols, modified food starch, acrylic polymers and mixtures of acrylic polymers with cellulose ethers such as Eudragit® EPO, Eudragit® RD100, and Eudragit® E100; cellulose acetate phthalate; sepifilms such as mixtures of HPMC and stearic acid, cyclodextrins, and mixtures of these materials. In other embodiments, additional taste-masking materials contemplated are those described in U.S. Pat. Nos. 4,851,226, 5,075,114, and 5,876,759, each of which is hereby incorporated by reference in its entirety.

In various embodiments, the excipient may include a pH modifier. In certain embodiments, the pH modifier may include sodium carbonate or sodium bicarbonate. In other embodiments, an antioxidant such as BHT or BHA is utilized.

The weight fraction of the excipient or combination of excipients in the pharmaceutical composition may be about 98% or less, about 95% or less, about 90% or less, about 85% or less, about 80% or less, about 75% or less, about 70% or less, about 65% or less, about 60% or less, about 55% or less, about 50% or less, about 45% or less, about 40% or less, about 35% or less, about 30% or less, about 25% or less, about 20% or less, about 15% or less, about 10% or less, about 5% or less, about 2%, or about 1% or less of the total weight of the pharmaceutical composition.

(h) Protectants

A mineral, nutrient or drug may be modified with a protectant such that its solubility is increased at higher pH levels than the unmodified compound. In one embodiment, the protectant may be an organic acid, an amino acid, a fatty acid, or a protein. For example, a mineral complexed or chelated with an organic acid, such as lactic acid or gluconic acid, is more soluble at neutral pH than the inorganic salts of the mineral (see section l(c) for more examples of organic mineral salts or chelates). Likewise, a drug may be complexed with an organic acid, an amino acid, or a fatty acid to generate a pharmaceutically acceptable salt, such as citrate, glutamate, lactate, malate, palmitate, tartrate, and the like. Methods to make organic mineral salts or pharmaceutically acceptable salts of biologically active agents are well known in the art.

In another embodiment, the protectant may be a coating or encapsulation such that the nutrient or drug may be absorbed throughout the intestinal tract independent of pH. The protectant coating may be a polymer, a protein, a lipid, and so forth, as detailed in section III.

In yet another embodiment, the nutrient or drug may be part of a multiple-component or multiple-crystalline composition, whereby the different crystalline assemblies may afford improved drug solubility, dissolution rate, stability and bioavailability. The principles of crystal engineering may be applied to form multiple-crystalline compositions using cocrystal formers that are complementary in the sense of supramolecular chemistry. The cocrystal formers may be, but are not limited to, solvent molecules, other drug molecules, GRAS compounds, or approved food additives. Pharmaceutical molecules or ions are inherently predisposed for such crystal engineering studies since they already contain molecular recognition sites that bind selectively to biomolecules, and thus, are prone to supramolecular self-assembly. Examples of the groups commonly found in drug molecules that are capable of forming supramolecular synthons include, but are not limited to, acids, amides, aliphatic nitrogen bases, unsaturated aromatic nitrogen bases (e.g. pyridines, imidazoles), amines, alcohols, halogens, sulfones, nitro groups, S-heterocycles, N-heterocycles (saturated or unsaturated), and O-heterocycles.

(i) Exemplary Formulations

Any of the pharmaceutical ingredients detailed in I(a) to (g) may be combined together to form pharmaceutical compositions of the invention. As will be appreciated by a skilled artisan, the choice of particular ingredients and their amounts will depend greatly upon the intended use of pharmaceutical composition. In this context, for example, if the pharmaceutical composition is administered to a subject to prevent or treat anemia, it will generally include an iron source. By way of further example, if the pharmaceutical composition is administered to a subject to prevent or treat osteoporosis, it will generally include a calcium source.

Suitable non-limiting examples of formulations are detailed in tables A to C below. In this context, iteration of suitable formulations includes the first agent on each line in combination with the second agent on each line. In table A, an agent that increases pH (i.e., first agent) may be combined with an agent that decreases pH (i.e., second agent).

TABLE A

| First Agent | Second Agent |
|---|---|
| Omeprazole | Phenylacetic acid |
| Omeprazole | Embonic acid |
| Omeprazole | Methanesulfonic acid |
| Omeprazole | Ethanesulfonic acid |
| Omeprazole | Benzenesulfonic acid |
| Omeprazole | Pantothenic acid |
| Omeprazole | Toluenesulfonic |
| Omeprazole | Galactaric acid |
| Omeprazole | Algenic acid |
| Omeprazole | Formic acid |
| Omeprazole | Acetic acid |
| Omeprazole | Propionic acid |
| Omeprazole | Succinic acid |
| Omeprazole | Glycolic acid |
| Omeprazole | Lactic acid |
| Omeprazole | Malic acid |
| Omeprazole | Tartaric acid |
| Omeprazole | Citric acid |
| Omeprazole | Ascorbic acid |
| Omeprazole | Glucuronic acid |
| Omeprazole | Maleic acid |
| Omeprazole | Fumaric acid |
| Omeprazole | Pyruvic acid |
| Omeprazole | Aspartic acid |
| Omeprazole | Glutamic acid |
| Omeprazole | Benzoic acid |
| Omeprazole | Anthronilic acid |
| Omeprazole | Mesylic acid |
| Omeprazole | Stearic acid |
| Omeprazole | Salicyclic acid |
| Omeprazole | p-hydroxybenzoic acid |
| Hydroxyomeprazole | Phenylacetic acid |
| Hydroxyomeprazole | Embonic acid |
| Hydroxyomeprazole | Methanesulfonic acid |
| Hydroxyomeprazole | Ethanesulfonic acid |
| Hydroxyomeprazole | Benzenesulfonic acid |
| Hydroxyomeprazole | Pantothenic acid |
| Hydroxyomeprazole | Toluenesulfonic acid |
| Hydroxyomeprazole | Galactaric acid |
| Hydroxyomeprazole | Algenic acid |
| Hydroxyomeprazole | Formic acid |
| Hydroxyomeprazole | Acetic acid |
| Hydroxyomeprazole | Propionic acid |
| Hydroxyomeprazole | Succinic acid |
| Hydroxyomeprazole | Glycolic acid |
| Hydroxyomeprazole | Lactic acid |
| Hydroxyomeprazole | Malic acid |
| Hydroxyomeprazole | Tartaric acid |
| Hydroxyomeprazole | Citric acid |
| Hydroxyomeprazole | Ascorbic acid |
| Hydroxyomeprazole | Glucuronic acid |
| Hydroxyomeprazole | Maleic acid |
| Hydroxyomeprazole | Fumaric acid |
| Hydroxyomeprazole | Pyruvic acid |
| Hydroxyomeprazole | Aspartic acid |
| Hydroxyomeprazole | Glutamic acid |
| Hydroxyomeprazole | Benzoic acid |
| Hydroxyomeprazole | Anthronilic acid |
| Hydroxyomeprazole | Mesylic acid |
| Hydroxyomeprazole | Stearic acid |
| Hydroxyomeprazole | Salicyclic acid |
| Hydroxyomeprazole | p-hydroxybenzoic acid |
| Esomeprazole | Phenylacetic acid |
| Esomeprazole | Embonic acid |
| Esomeprazole | Methanesulfonic acid |
| Esomeprazole | Ethanesulfonic acid |
| Esomeprazole | Benzenesulfonic acid |
| Esomeprazole | Pantothenic acid |
| Esomeprazole | Toluenesulfonic acid |
| Esomeprazole | Galactaric acid |
| Esomeprazole | Algenic acid |
| Esomeprazole | Formic acid |
| Esomeprazole | Acetic acid |
| Esomeprazole | Propionic acid |
| Esomeprazole | Succinic acid |
| Esomeprazole | Glycolic acid |
| Esomeprazole | Lactic acid |
| Esomeprazole | Malic acid |
| Esomeprazole | Tartaric acid |
| Esomeprazole | Citric acid |
| Esomeprazole | Ascorbic acid |
| Esomeprazole | Glucuronic acid |
| Esomeprazole | Maleic acid |
| Esomeprazole | Fumaric acid |
| Esomeprazole | Pyruvic acid |
| Esomeprazole | Aspartic acid |
| Esomeprazole | Glutamic acid |
| Esomeprazole | Benzoic acid |
| Esomeprazole | Anthronilic acid |
| Esomeprazole | Mesylic acid |
| Esomeprazole | Stearic acid |
| Esomeprazole | Salicyclic acid |
| Esomeprazole | p-hydroxybenzoic acid |
| Tenatopruzole | Phenylacetic acid |
| Tenatopruzole | Embonic acid |
| Tenatopruzole | Methanesulfonic acid |
| Tenatopruzole | Ethanesulfonic acid |
| Tenatopruzole | Benzenesulfonic acid |
| Tenatopruzole | Pantothenic acid |
| Tenatopruzole | Toluenesulfonic acid |
| Tenatopruzole | Galactaric acid |
| Tenatopruzole | Algenic acid |
| Tenatopruzole | Formic acid |
| Tenatopruzole | Acetic acid |
| Tenatopruzole | Propionic acid |
| Tenatopruzole | Succinic acid |
| Tenatopruzole | Glycolic acid |
| Tenatopruzole | Lactic acid |
| Tenatopruzole | Malic acid |
| Tenatopruzole | Tartaric acid |
| Tenatopruzole | Citric acid |
| Tenatopruzole | Ascorbic acid |
| Tenatopruzole | Glucuronic acid |
| Tenatopruzole | Maleic acid |
| Tenatopruzole | Fumaric acid |
| Tenatopruzole | Pyruvic acid |
| Tenatopruzole | Aspartic acid |
| Tenatopruzole | Glutamic acid |
| Tenatopruzole | Benzoic acid |
| Tenatopruzole | Anthronilic acid |
| Tenatopruzole | Mesylic acid |
| Tenatopruzole | Stearic acid |
| Tenatopruzole | Salicyclic acid |
| Tenatopruzole | p-hydroxybenzoic acid |
| Lansoprazole | Phenylacetic acid |
| Lansoprazole | Embonic acid |
| Lansoprazole | Methanesulfonic acid |
| Lansoprazole | Ethanesulfonic acid |
| Lansoprazole | Benzenesulfonic acid |
| Lansoprazole | Pantothenic acid |
| Lansoprazole | Toluenesulfonic acid |
| Lansoprazole | Galactaric acid |
| Lansoprazole | Algenic acid |
| Lansoprazole | Formic acid |
| Lansoprazole | Acetic acid |
| Lansoprazole | Propionic acid |
| Lansoprazole | Succinic acid |
| Lansoprazole | Glycolic acid |
| Lansoprazole | Lactic acid |
| Lansoprazole | Malic acid |
| Lansoprazole | Tartaric acid |
| Lansoprazole | Citric acid |
| Lansoprazole | Ascorbic acid |
| Lansoprazole | Glucuronic acid |
| Lansoprazole | Maleic acid |
| Lansoprazole | Fumaric acid |
| Lansoprazole | Pyruvic acid |
| Lansoprazole | Aspartic acid |
| Lansoprazole | Glutamic acid |
| Lansoprazole | Benzoic acid |
| Lansoprazole | Anthronilic acid |
| Lansoprazole | Mesylic acid |
| Lansoprazole | Stearic acid |
| Lansoprazole | Salicyclic acid |
| Lansoprazole | p-hydroxybenzoic acid |
| Pantoprazole | Phenylacetic acid |
| Pantoprazole | Embonic acid |
| Pantoprazole | Methanesulfonic acid |
| Pantoprazole | Ethanesulfonic acid |
| Pantoprazole | Benzenesulfonic acid |
| Pantoprazole | Pantothenic acid |
| Pantoprazole | Toluenesufonic acid |
| Pantoprazole | Galactaric acid |
| Pantoprazole | Algenic acid |
| Pantoprazole | Formic acid |
| Pantoprazole | Acetic acid |
| Pantoprazole | Propionic acid |
| Pantoprazole | Succinic acid |
| Pantoprazole | Glycolic acid |
| Pantoprazole | Lactic acid |
| Pantoprazole | Malic acid |
| Pantoprazole | Tartaric acid |
| Pantoprazole | Citric acid |
| Pantoprazole | Ascorbic acid |
| Pantoprazole | Glucuronic acid |
| Pantoprazole | Maleic acid |
| Pantoprazole | Fumaric acid |
| Pantoprazole | Pyruvic acid |
| Pantoprazole | Aspartic acid |
| Pantoprazole | Glutamic acid |
| Pantoprazole | Benzoic acid |
| Pantoprazole | Anthronilic acid |
| Pantoprazole | Mesylic acid |
| Pantoprazole | Stearic acid |
| Pantoprazole | Salicyclic acid |
| Pantoprazole | p-hydroxybenzoic acid |
| Rabeprazole | Phenylacetic acid |
| Rabeprazole | Embonic acid |
| Rabeprazole | Methanesulfonic acid |
| Rabeprazole | Ethanesulfonic acid |
| Rabeprazole | Benzenesulfonic acid |
| Rabeprazole | Pantothenic acid |
| Rabeprazole | Toluenesulfonic acid |
| Rabeprazole | Galactaric acid |
| Rabeprazole | Algenic acid |
| Rabeprazole | Formic acid |
| Rabeprazole | Acetic acid |
| Rabeprazole | Propionic acid |
| Rabeprazole | Succinic acid |
| Rabeprazole | Glycolic acid |
| Rabeprazole | Lactic acid |
| Rabeprazole | Malic acid |
| Rabeprazole | Tartaric acid |
| Rabeprazole | Citric acid |
| Rabeprazole | Ascorbic acid |
| Rabeprazole | Glucuronic acid |
| Rabeprazole | Maleic acid |
| Rabeprazole | Fumaric acid |
| Rabeprazole | Pyruvic acid |
| Rabeprazole | Aspartic acid |
| Rabeprazole | Glutamic acid |
| Rabeprazole | Benzoic acid |
| Rabeprazole | Anthronilic acid |
| Rabeprazole | Mesylic acid |
| Rabeprazole | Stearic acid |
| Rabeprazole | Salicyclic acid |
| Rabeprazole | p-hydroxybenzoic acid |
| Dontoprazole | Phenylacetic acid |
| Dontoprazole | Embonic acid |
| Dontoprazole | Methanesulfonic acid |
| Dontoprazole | Ethanesulfonic acid |
| Dontoprazole | Benzenesulfonic acid |
| Dontoprazole | Pantothenic acid |
| Dontoprazole | Toluenesulfonic acid |
| Dontoprazole | Galactaric acid |
| Dontoprazole | Algenic acid |
| Dontoprazole | Formic acid |
| Dontoprazole | Acetic acid |
| Dontoprazole | Propionic acid |
| Dontoprazole | Succinic acid |
| Dontoprazole | Glycolic acid |
| Dontoprazole | Lactic acid |
| Dontoprazole | Malic acid |
| Dontoprazole | Tartaric acid |
| Dontoprazole | Citric acid |
| Dontoprazole | Ascorbic acid |
| Dontoprazole | Glucuronic acid |
| Dontoprazole | Maleic acid |
| Dontoprazole | Fumaric acid |
| Dontoprazole | Pyruvic acid |
| Dontoprazole | Aspartic acid |
| Dontoprazole | Glutamic acid |
| Dontoprazole | Benzoic acid |
| Dontoprazole | Anthronilic acid |
| Dontoprazole | Mesylic acid |
| Dontoprazole | Stearic acid |
| Dontoprazole | Salicyclic acid |
| Dontoprazole | p-hydroxybenzoic acid |
| Habeprazole | Phenylacetic acid |
| Habeprazole | Embonic acid |
| Habeprazole | Methanesulfonic acid |
| Habeprazole | Ethanesulfonic acid |
| Habeprazole | Benzenesulfonic acid |
| Habeprazole | Pantothenic acid |
| Habeprazole | Toluenesulfonic acid |
| Habeprazole | Galactaric acid |
| Habeprazole | Algenic acid |
| Habeprazole | Formic acid |
| Habeprazole | Acetic acid |
| Habeprazole | Propionic acid |
| Habeprazole | Succinic acid |
| Habeprazole | Glycolic acid |
| Habeprazole | Lactic acid |
| Habeprazole | Malic acid |
| Habeprazole | Tartaric acid |
| Habeprazole | Citric acid |
| Habeprazole | Ascorbic acid |
| Habeprazole | Glucuronic acid |
| Habeprazole | Maleic acid |
| Habeprazole | Fumaric acid |
| Habeprazole | Pyruvic acid |
| Habeprazole | Aspartic acid |
| Habeprazole | Glutamic acid |
| Habeprazole | Benzoic acid |
| Habeprazole | Anthronilic acid |
| Habeprazole | Mesylic acid |
| Habeprazole | Stearic acid |
| Habeprazole | Salicyclic acid |
| Habeprazole | p-hydroxybenzoic acid |
| Perprazole | Phenylacetic acid |
| Perprazole | Embonic acid |
| Perprazole | Methanesulfonic acid |
| Perprazole | Ethanesulfonic acid |
| Perprazole | Benzenesulfonic acid |
| Perprazole | Pantothenic acid |
| Perprazole | Toluenesulfonic acid |
| Perprazole | Galactaric acid |
| Perprazole | Algenic acid |
| Perprazole | Formic acid |
| Perprazole | Acetic acid |
| Perprazole | Propionic acid |
| Perprazole | Succinic acid |
| Perprazole | Glycolic acid |
| Perprazole | Lactic acid |
| Perprazole | Malic acid |
| Perprazole | Tartaric acid |
| Perprazole | Citric acid |
| Perprazole | Ascorbic acid |
| Perprazole | Glucuronic acid |
| Perprazole | Maleic acid |
| Perprazole | Fumaric acid |
| Perprazole | Pyruvic acid |
| Perprazole | Aspartic acid |
| Perprazole | Glutamic acid |
| Perprazole | Benzoic acid |
| Perprazole | Anthronilic acid |
| Perprazole | Mesylic acid |
| Perprazole | Stearic acid |
| Perprazole | Salicyclic acid |
| Perprazole | p-hydroxybenzoic acid |
| Ransoprazole | Phenylacetic acid |
| Ransoprazole | Embonic acid |
| Ransoprazole | Methanesulfonic acid |
| Ransoprazole | Ethanesulfonic acid |
| Ransoprazole | Benzenesulfonic acid |
| Ransoprazole | Pantothenic acid |
| Ransoprazole | Toluenesulfonic acid |
| Ransoprazole | Galactaric acid |
| Ransoprazole | Algenic acid |
| Ransoprazole | Formic acid |
| Ransoprazole | Acetic acid |
| Ransoprazole | Propionic acid |
| Ransoprazole | Succinic acid |
| Ransoprazole | Glycolic acid |
| Ransoprazole | Lactic acid |
| Ransoprazole | Malic acid |
| Ransoprazole | Tartaric acid |
| Ransoprazole | Citric acid |
| Ransoprazole | Ascorbic acid |
| Ransoprazole | Glucuronic acid |
| Ransoprazole | Maleic acid |
| Ransoprazole | Fumaric acid |
| Ransoprazole | Pyruvic acid |
| Ransoprazole | Aspartic acid |
| Ransoprazole | Glutamic acid |
| Ransoprazole | Benzoic acid |
| Ransoprazole | Anthronilic acid |
| Ransoprazole | Mesylic acid |
| Ransoprazole | Stearic acid |
| Ransoprazole | Salicyclic acid |
| Ransoprazole | p-hydroxybenzoic acid |
| Pariprazole | Phenylacetic acid |
| Pariprazole | Embonic acid |
| Pariprazole | Methanesulfonic acid |
| Pariprazole | Ethanesulfonic acid |
| Pariprazole | Benzenesulfonic acid |
| Pariprazole | Pantothenic acid |
| Pariprazole | Toluenesulfonic acid |
| Pariprazole | Galactaric acid |
| Pariprazole | Algenic acid |
| Pariprazole | Formic acid |
| Pariprazole | Acetic acid |
| Pariprazole | Propionic acid |
| Pariprazole | Succinic acid |
| Pariprazole | Glycolic acid |
| Pariprazole | Lactic acid |
| Pariprazole | Malic acid |
| Pariprazole | Tartaric acid |
| Pariprazole | Citric acid |
| Pariprazole | Ascorbic acid |
| Pariprazole | Glucuronic acid |
| Pariprazole | Maleic acid |
| Pariprazole | Fumaric acid |
| Pariprazole | Pyruvic acid |
| Pariprazole | Aspartic acid |
| Pariprazole | Glutamic acid |
| Pariprazole | Benzoic acid |
| Pariprazole | Anthronilic acid |
| Pariprazole | Mesylic acid |
| Pariprazole | Stearic acid |
| Pariprazole | Salicyclic acid |
| Pariprazole | p-hydroxybenzoic acid |
| Leminoprazole | Phenylacetic acid |
| Leminoprazole | Embonic acid |
| Leminoprazole | Methanesulfonic acid |
| Leminoprazole | Ethanesulfonic acid |
| Leminoprazole | Benzenesulfonic acid |
| Leminoprazole | Pantothenic acid |
| Leminoprazole | Toluenesulfonic acid |
| Leminoprazole | Galactaric acid |
| Leminoprazole | Algenic acid |
| Leminoprazole | Formic acid |
| Leminoprazole | Acetic acid |
| Leminoprazole | Propionic acid |
| Leminoprazole | Succinic acid |
| Leminoprazole | Glycolic acid |
| Leminoprazole | Lactic acid |
| Leminoprazole | Malic acid |
| Leminoprazole | Tartaric acid |
| Leminoprazole | Citric acid |
| Leminoprazole | Ascorbic acid |
| Leminoprazole | Glucuronic acid |
| Leminoprazole | Maleic acid |
| Leminoprazole | Fumaric acid |
| Leminoprazole | Pyruvic acid |
| Leminoprazole | Aspartic acid |
| Leminoprazole | Glutamic acid |
| Leminoprazole | Benzoic acid |
| Leminoprazole | Anthronilic acid |
| Leminoprazole | Mesylic acid |
| Leminoprazole | Stearic acid |
| Leminoprazole | Salicyclic acid |
| Leminoprazole | p-hydroxybenzoic acid |
| Cimetidine | Phenylacetic acid |
| Cimetidine | Embonic acid |
| Cimetidine | Methanesulfonic acid |
| Cimetidine | Ethanesulfonic acid |
| Cimetidine | Benzenesulfonic acid |
| Cimetidine | Pantothenic acid |
| Cimetidine | Toluenesulfonic acid |
| Cimetidine | Galactaric acid |
| Cimetidine | Algenic acid |
| Cimetidine | Formic acid |
| Cimetidine | Acetic acid |
| Cimetidine | Propionic acid |
| Cimetidine | Succinic acid |
| Cimetidine | Glycolic acid |
| Cimetidine | Lactic acid |
| Cimetidine | Malic acid |
| Cimetidine | Tartaric acid |
| Cimetidine | Citric acid |
| Cimetidine | Ascorbic acid |
| Cimetidine | Glucuronic acid |
| Cimetidine | Maleic acid |
| Cimetidine | Fumaric acid |
| Cimetidine | Pyruvic acid |
| Cimetidine | Aspartic acid |
| Cimetidine | Glutamic acid |
| Cimetidine | Benzoic acid |
| Cimetidine | Anthronilic acid |
| Cimetidine | Mesylic acid |
| Cimetidine | Stearic acid |
| Cimetidine | Salicyclic acid |
| Cimetidine | p-hydroxybenzoic acid |
| Famotidine | Phenylacetic acid |
| Famotidine | Embonic acid |
| Famotidine | Methanesulfonic acid |
| Famotidine | Ethanesulfonic acid |
| Famotidine | Benzenesulfonic acid |
| Famotidine | Pantothenic acid |
| Famotidine | Toluenesulfonic acid |
| Famotidine | Galactaric acid |
| Famotidine | Algenic acid |
| Famotidine | Formic acid |
| Famotidine | Acetic acid |
| Famotidine | Propionic acid |
| Famotidine | Succinic acid |
| Famotidine | Glycolic acid |
| Famotidine | Lactic acid |
| Famotidine | Malic acid |
| Famotidine | Tartaric acid |
| Famotidine | Citric acid |
| Famotidine | Ascorbic acid |
| Famotidine | Glucuronic acid |
| Famotidine | Maleic acid |
| Famotidine | Fumaric acid |
| Famotidine | Pyruvic acid |
| Famotidine | Aspartic acid |
| Famotidine | Glutamic acid |
| Famotidine | Benzoic acid |
| Famotidine | Anthronilic acid |
| Famotidine | Mesylic acid |
| Famotidine | Stearic acid |
| Famotidine | Salicyclic acid |
| Famotidine | p-hydroxybenzoic acid |
| Nizatidine | Phenylacetic |
| Nizatidine | Embonic acid |
| Nizatidine | Methanesulfonic acid |
| Nizatidine | Ethanesulfonic acid |
| Nizatidine | Benzenesulfonic acid |
| Nizatidine | Pantothenic acid |
| Nizatidine | Toluenesulfonic acid |
| Nizatidine | Galactaric acid |
| Nizatidine | Algenic acid |
| Nizatidine | Formic acid |
| Nizatidine | Acetic acid |
| Nizatidine | Propionic acid |
| Nizatidine | Succinic acid |
| Nizatidine | Glycolic acid |
| Nizatidine | Lactic acid |
| Nizatidine | Malic acid |
| Nizatidine | Tartaric acid |
| Nizatidine | Citric acid |
| Nizatidine | Ascorbic acid |
| Nizatidine | Glucuronic acid |
| Nizatidine | Maleic acid |
| Nizatidine | Fumaric acid |
| Nizatidine | Pyruvic acid |
| Nizatidine | Aspartic acid |
| Nizatidine | Glutamic acid |
| Nizatidine | Benzoic acid |
| Nizatidine | Anthronilic acid |
| Nizatidine | Mesylic acid |
| Nizatidine | Stearic acid |
| Nizatidine | Salicyclic acid |
| Nizatidine | p-hydroxybenzoic acid |
| Ranitidine | Phenylacetic acid |
| Ranitidine | Embonic acid |
| Ranitidine | Methanesulfonic acid |
| Ranitidine | Ethanesulfonic acid |
| Ranitidine | Benzenesulfonic acid |
| Ranitidine | Pantothenic acid |
| Ranitidine | Toluenesulfonic acid |
| Ranitidine | Galactaric acid |
| Ranitidine | Algenic acid |
| Ranitidine | Formic acid |
| Ranitidine | Acetic acid |
| Ranitidine | Propionic acid |
| Ranitidine | Succinic acid |
| Ranitidine | Glycolic acid |
| Ranitidine | Lactic acid |
| Ranitidine | Malic acid |
| Ranitidine | Tartaric acid |
| Ranitidine | Citric acid |
| Ranitidine | Ascorbic acid |
| Ranitidine | Glucuronic acid |
| Ranitidine | Maleic acid |
| Ranitidine | Fumaric acid |
| Ranitidine | Pyruvic acid |
| Ranitidine | Aspartic acid |
| Ranitidine | Glutamic acid |
| Ranitidine | Benzoic acid |
| Ranitidine | Anthronilic acid |
| Ranitidine | Mesylic acid |
| Ranitidine | Stearic acid |
| Ranitidine | Salicyclic acid |
| Ranitidine | p-hydroxybenzoic acid |

Any of the formulations detailed in Table A may further include a vitamin, mineral, drug, excipient, buffering agent, or any combination of these additional ingredients. In one embodiment, the vitamin may be selected from vitamin C, vitamin A, vitamin E, vitamin K, vitamin D, vitamin B group, including vitamin B12, riboflavin, niacin, vitamin B6, folic acid, pyridoxine, thiamine, pantothenic acid, and biotin. In an exemplary embodiment, the vitamin is vitamin B12, vitamin C, vitamin D, or vitamin E. In another embodiment, the mineral may be selected from calcium, chromium, copper, iodine, iron, magnesium, manganese, molybdenum, phosphorus, potassium, zinc, and selenium. In an exemplary embodiment, the mineral is calcium, iron or zinc. By way of non-limiting example, an exemplary formulation may include a proton pump inhibitor, an organic acid selected from succinic acid, ascorbic acid, and glutamic acid, and a vitamin selected from vitamin B12, vitamin C, and vitamin E. Optionally, this formulation may include calcium and/or iron. By way of further non-limiting, an exemplary formulation may include a proton pump inhibitor, an organic acid selected from ascorbic acid and succinic acid, and calcium and/or iron.

Table B represents exemplary formulations having an agent that increases pH (i.e., first agent) combined with a mineral (i.e., second agent).

TABLE B

| First Agent | Second Agent |
|---|---|
| Omeprazole | Calcium |
| Omeprazole | Chromium |
| Omeprazole | Copper |
| Omeprazole | Iodine |
| Omeprazole | Iron |
| Omeprazole | Magnesium |
| Omeprazole | Manganese |
| Omeprazole | Molybdenum |
| Omeprazole | Phosphorus |
| Omeprazole | Potassium |
| Omeprazole | Selenium |
| Omeprazole | Zinc |
| Hydroxyomeprazole | Calcium |
| Hydroxyomeprazole | Chromium |
| Hydroxyomeprazole | Copper |
| Hydroxyomeprazole | Iodine |
| Hydroxyomeprazole | Iron |
| Hydroxyomeprazole | Magnesium |
| Hydroxyomeprazole | Manganese |
| Hydroxyomeprazole | Molybdenum |
| Hydroxyomeprazole | Phosphorus |
| Hydroxyomeprazole | Potassium |
| Hydroxyomeprazole | Selenium |
| Hydroxyomeprazole | Zinc |
| Esomeprazole | Calcium |
| Esomeprazole | Chromium |
| Esomeprazole | Copper |
| Esomeprazole | Iodine |
| Esomeprazole | Iron |
| Esomeprazole | Magnesium |
| Esomeprazole | Manganese |
| Esomeprazole | Molybdenum |
| Esomeprazole | Phosphorus |
| Esomeprazole | Potassium |
| Esomeprazole | Selenium |
| Esomeprazole | Zinc |
| Tenatopruzole | Calcium |
| Tenatopruzole | Chromium |
| Tenatopruzole | Copper |
| Tenatopruzole | Iodine |
| Tenatopruzole | Iron |
| Tenatopruzole | Magnesium |
| Tenatopruzole | Manganese |
| Tenatopruzole | Molybdenum |
| Tenatopruzole | Phosphorus |
| Tenatopruzole | Potassium |
| Tenatopruzole | Selenium |
| Tenatopruzole | Zinc |
| Lansoprazole | Calcium |
| Lansoprazole | Chromium |
| Lansoprazole | Copper |
| Lansoprazole | Iodine |
| Lansoprazole | Iron |
| Lansoprazole | Magnesium |
| Lansoprazole | Manganese |
| Lansoprazole | Molybdenum |
| Lansoprazole | Phosphorus |
| Lansoprazole | Potassium |
| Lansoprazole | Selenium |
| Lansoprazole | Zinc |
| Pantoprazole | Calcium |
| Pantoprazole | Chromium |
| Pantoprazole | Copper |
| Pantoprazole | Iodine |
| Pantoprazole | Iron |
| Pantoprazole | Magnesium |
| Pantoprazole | Manganese |
| Pantoprazole | Molybdenum |
| Pantoprazole | Phosphorus |
| Pantoprazole | Potassium |
| Pantoprazole | Selenium |
| Pantoprazole | Zinc |
| Rabeprazole | Calcium |
| Rabeprazole | Chromium |
| Rabeprazole | Copper |
| Rabeprazole | Iodine |
| Rabeprazole | Iron |
| Rabeprazole | Magnesium |
| Rabeprazole | Manganese |
| Rabeprazole | Molybdenum |
| Rabeprazole | Phosphorus |
| Rabeprazole | Potassium |
| Rabeprazole | Selenium |
| Rabeprazole | Zinc |
| Dontoprazole | Calcium |
| Dontoprazole | Chromium |
| Dontoprazole | Copper |
| Dontoprazole | Iodine |
| Dontoprazole | Iron |
| Dontoprazole | Magnesium |
| Dontoprazole | Manganese |
| Dontoprazole | Molybdenum |
| Dontoprazole | Phosphorus |
| Dontoprazole | Potassium |
| Dontoprazole | Selenium |
| Dontoprazole | Zinc |
| Habeprazole | Calcium |
| Habeprazole | Chromium |
| Habeprazole | Copper |
| Habeprazole | Iodine |
| Habeprazole | Iron |
| Habeprazole | Magnesium |
| Habeprazole | Manganese |
| Habeprazole | Molybdenum |
| Habeprazole | Phosphorus |
| Habeprazole | Potassium |
| Habeprazole | Selenium |
| Habeprazole | Zinc |
| Perprazole | Calcium |
| Perprazole | Chromium |
| Perprazole | Copper |
| Perprazole | Iodine |
| Perprazole | Iron |
| Perprazole | Magnesium |
| Perprazole | Manganese |
| Perprazole | Molybdenum |
| Perprazole | Phosphorus |
| Perprazole | Potassium |
| Perprazole | Selenium |
| Perprazole | Zinc |
| Ransoprazole | Calcium |
| Ransoprazole | Chromium |
| Ransoprazole | Copper |
| Ransoprazole | Iodine |
| Ransoprazole | Iron |
| Ransoprazole | Magnesium |
| Ransoprazole | Manganese |
| Ransoprazole | Molybdenum |
| Ransoprazole | Phosphorus |
| Ransoprazole | Potassium |
| Ransoprazole | Selenium |
| Ransoprazole | Zinc |
| Pariprazole | Calcium |
| Pariprazole | Chromium |
| Pariprazole | Copper |
| Pariprazole | Iodine |
| Pariprazole | Iron |
| Pariprazole | Magnesium |
| Pariprazole | Manganese |
| Pariprazole | Molybdenum |
| Pariprazole | Phosphorus |
| Pariprazole | Potassium |
| Pariprazole | Selenium |
| Pariprazole | Zinc |
| Leminoprazole | Calcium |
| Leminoprazole | Chromium |
| Leminoprazole | Copper |
| Leminoprazole | Iodine |
| Leminoprazole | Iron |
| Leminoprazole | Magnesium |
| Leminoprazole | Manganese |
| Leminoprazole | Molybdenum |
| Leminoprazole | Phosphorus |
| Leminoprazole | Potassium |
| Leminoprazole | Selenium |
| Leminoprazole | Zinc |
| Cimetidine | Calcium |
| Cimetidine | Chromium |
| Cimetidine | Copper |
| Cimetidine | Iodine |
| Cimetidine | Iron |
| Cimetidine | Magnesium |
| Cimetidine | Manganese |
| Cimetidine | Molybdenum |
| Cimetidine | Phosphorus |
| Cimetidine | Potassium |
| Cimetidine | Selenium |
| Cimetidine | Zinc |
| Famotidine | Calcium |
| Famotidine | Chromium |
| Famotidine | Copper |
| Famotidine | Iodine |
| Famotidine | Iron |
| Famotidine | Magnesium |
| Famotidine | Manganese |
| Famotidine | Molybdenum |
| Famotidine | Phosphorus |
| Famotidine | Potassium |
| Famotidine | Selenium |
| Famotidine | Zinc |
| Nizatidine | Calcium |
| Nizatidine | Chromium |
| Nizatidine | Copper |
| Nizatidine | Iodine |
| Nizatidine | Iron |
| Nizatidine | Magnesium |
| Nizatidine | Manganese |
| Nizatidine | Molybdenum |
| Nizatidine | Phosphorus |
| Nizatidine | Potassium |
| Nizatidine | Selenium |
| Nizatidine | Zinc |
| Ranitidine | Calcium |
| Ranitidine | Chromium |
| Ranitidine | Copper |
| Ranitidine | Iodine |
| Ranitidine | Iron |
| Ranitidine | Magnesium |
| Ranitidine | Manganese |
| Ranitidine | Molybdenum |
| Ranitidine | Phosphorus |
| Ranitidine | Potassium |
| Ranitidine | Selenium |
| Ranitidine | Zinc |

Any of the formulations detailed in Table B may further include a vitamin, organic acid, drug, excipient, buffering agent, or any combination of these additional ingredients. In one embodiment, the vitamin may be selected from vitamin C, vitamin A, vitamin E, vitamin B12, vitamin K, riboflavin, niacin, vitamin D, vitamin B6, folic acid, pyridoxine, thiamine, pantothenic acid, and biotin. In an exemplary embodiment, the vitamin is vitamin B12, vitamin C, vitamin D, and vitamin E. By way of non-limiting example, an exemplary formulation may include a proton pump inhibitor, calcium or iron, and a vitamin selected from vitamin B12, vitamin C, vitamin D, and vitamin E. Optionally, this formulation may include an organic acid selected from succinic acid, ascorbic acid and glutamic acid.

Table C represents exemplary formulations having an agent that increases pH (i.e., first agent) combined with a vitamin (i.e., second agent).

TABLE C

| First Agent | Second Agent |
|---|---|
| Omeprazole | Vitamin C |
| Omeprazole | Vitamin A |
| Omeprazole | Vitamin E |
| Omeprazole | Vitamin B12 |
| Omeprazole | Vitamin K |
| Omeprazole | Riboflavin |
| Omeprazole | Niacin |
| Omeprazole | Vitamin D |
| Omeprazole | Vitamin B6 |
| Omeprazole | Folic acid |
| Omeprazole | Pyridoxine |
| Omeprazole | Thiamine |
| Omeprazole | Pantothenic acid |
| Omeprazole | Biotin |
| Hydroxyomeprazole | Vitamin C |
| Hydroxyomeprazole | Vitamin A |
| Hydroxyomeprazole | Vitamin E |
| Hydroxyomeprazole | Vitamin B12 |
| Hydroxyomeprazole | Vitamin K |
| Hydroxyomeprazole | Riboflavin |
| Hydroxyomeprazole | Niacin |
| Hydroxyomeprazole | Vitamin D |
| Hydroxyomeprazole | Vitamin B6 |
| Hydroxyomeprazole | Folic acid |
| Hydroxyomeprazole | Pyridoxine |
| Hydroxyomeprazole | Thiamine |
| Hydroxyomeprazole | Pantothenic acid |
| Hydroxyomeprazole | Biotin |
| Esomeprazole | Vitamin C |
| Esomeprazole | Vitamin A |
| Esomeprazole | Vitamin E |
| Esomeprazole | Vitamin B12 |
| Esomeprazole | Vitamin K |
| Esomeprazole | Riboflavin |
| Esomeprazole | Niacin |
| Esomeprazole | Vitamin D |
| Esomeprazole | Vitamin B6 |
| Esomeprazole | Folic acid |
| Esomeprazole | Pyridoxine |
| Esomeprazole | Thiamine |
| Esomeprazole | Pantothenic acid |
| Esomeprazole | Biotin |
| Tenatopruzole | Vitamin C |
| Tenatopruzole | Vitamin A |
| Tenatopruzole | Vitamin E |
| Tenatopruzole | Vitamin B12 |
| Tenatopruzole | Vitamin K |
| Tenatopruzole | Riboflavin |
| Tenatopruzole | Niacin |
| Tenatopruzole | Vitamin D |
| Tenatopruzole | Vitamin B6 |
| Tenatopruzole | Folic acid |
| Tenatopruzole | Pyridoxine |
| Tenatopruzole | Thiamine |
| Tenatopruzole | Pantothenic acid |
| Tenatopruzole | Biotin |
| Lansoprazole | Vitamin C |
| Lansoprazole | Vitamin A |
| Lansoprazole | Vitamin E |
| Lansoprazole | Vitamin B12 |
| Lansoprazole | Vitamin K |
| Lansoprazole | Riboflavin |
| Lansoprazole | Niacin |
| Lansoprazole | Vitamin D |
| Lansoprazole | Vitamin B6 |
| Lansoprazole | Folic acid |
| Lansoprazole | Pyridoxine |
| Lansoprazole | Thiamine |
| Lansoprazole | Pantothenic acid |
| Lansoprazole | Biotin |
| Pantoprazole | Vitamin C |
| Pantoprazole | Vitamin A |
| Pantoprazole | Vitamin E |
| Pantoprazole | Vitamin B12 |
| Pantoprazole | Vitamin K |
| Pantoprazole | Riboflavin |
| Pantoprazole | Niacin |
| Pantoprazole | Vitamin D |
| Pantoprazole | Vitamin B6 |
| Pantoprazole | Folic acid |
| Pantoprazole | Pyridoxine |
| Pantoprazole | Thiamine |
| Pantoprazole | Pantothenic acid |
| Pantoprazole | Biotin |
| Rabeprazole | Vitamin C |
| Rabeprazole | Vitamin A |
| Rabeprazole | Vitamin E |
| Rabeprazole | Vitamin B12 |
| Rabeprazole | Vitamin K |
| Rabeprazole | Riboflavin |
| Rabeprazole | Niacin |
| Rabeprazole | Vitamin D |
| Rabeprazole | Vitamin B6 |
| Rabeprazole | Folic acid |
| Rabeprazole | Pyridoxine |
| Rabeprazole | Thiamine |
| Rabeprazole | Pantothenic acid |
| Rabeprazole | Biotin |
| Dontoprazole | Vitamin C |
| Dontoprazole | Vitamin A |
| Dontoprazole | Vitamin E |
| Dontoprazole | Vitamin B12 |
| Dontoprazole | Vitamin K |
| Dontoprazole | Riboflavin |
| Dontoprazole | Niacin |
| Dontoprazole | Vitamin D |
| Dontoprazole | Vitamin B6 |
| Dontoprazole | Folic acid |
| Dontoprazole | Pyridoxine |
| Dontoprazole | Thiamine |
| Dontoprazole | Pantothenic acid |
| Dontoprazole | Biotin |
| Habeprazole | Vitamin C |
| Habeprazole | Vitamin A |
| Habeprazole | Vitamin E |
| Habeprazole | Vitamin B12 |
| Habeprazole | Vitamin K |
| Habeprazole | Riboflavin |
| Habeprazole | Niacin |
| Habeprazole | Vitamin D |
| Habeprazole | Vitamin B6 |
| Habeprazole | Folic acid |
| Habeprazole | Pyridoxine |
| Habeprazole | Thiamine |
| Habeprazole | Pantothenic acid |
| Habeprazole | Biotin |
| Perprazole | Vitamin C |
| Perprazole | Vitamin A |
| Perprazole | Vitamin E |
| Perprazole | Vitamin B12 |
| Perprazole | Vitamin K |
| Perprazole | Riboflavin |
| Perprazole | Niacin |
| Perprazole | Vitamin D |
| Perprazole | Vitamin B6 |
| Perprazole | Folic acid |
| Perprazole | Pyridoxine |
| Perprazole | Thiamine |
| Perprazole | Pantothenic acid |
| Perprazole | Biotin |
| Ransoprazole | Vitamin C |
| Ransoprazole | Vitamin A |
| Ransoprazole | Vitamin E |
| Ransoprazole | Vitamin B12 |
| Ransoprazole | Vitamin K |
| Ransoprazole | Riboflavin |
| Ransoprazole | Niacin |
| Ransoprazole | Vitamin D |
| Ransoprazole | Vitamin B6 |
| Ransoprazole | Folic acid |
| Ransoprazole | Pyridoxine |
| Ransoprazole | Thiamine |
| Ransoprazole | Pantothenic acid |
| Ransoprazole | Biotin |
| Pariprazole | Vitamin C |
| Pariprazole | Vitamin A |
| Pariprazole | Vitamin E |
| Pariprazole | Vitamin B12 |
| Pariprazole | Vitamin K |
| Pariprazole | Riboflavin |
| Pariprazole | Niacin |
| Pariprazole | Vitamin D |
| Pariprazole | Vitamin B6 |
| Pariprazole | Folic acid |
| Pariprazole | Pyridoxine |
| Pariprazole | Thiamine |
| Pariprazole | Pantothenic acid |
| Pariprazole | Biotin |
| Leminoprazole | Vitamin C |
| Leminoprazole | Vitamin A |
| Leminoprazole | Vitamin E |
| Leminoprazole | Vitamin B12 |
| Leminoprazole | Vitamin K |
| Leminoprazole | Riboflavin |
| Leminoprazole | Niacin |
| Leminoprazole | Vitamin D |
| Leminoprazole | Vitamin B6 |
| Leminoprazole | Folic acid |
| Leminoprazole | Pyridoxine |
| Leminoprazole | Thiamine |
| Leminoprazole | Pantothenic acid |
| Leminoprazole | Biotin |
| Cimetidine | Vitamin C |
| Cimetidine | Vitamin A |
| Cimetidine | Vitamin E |
| Cimetidine | Vitamin B12 |
| Cimetidine | Vitamin K |
| Cimetidine | Riboflavin |
| Cimetidine | Niacin |
| Cimetidine | Vitamin D |
| Cimetidine | Vitamin B6 |
| Cimetidine | Folic acid |
| Cimetidine | Pyridoxine |
| Cimetidine | Thiamine |
| Cimetidine | Pantothenic acid |
| Cimetidine | Biotin |
| Famotidine | Vitamin C |
| Famotidine | Vitamin A |
| Famotidine | Vitamin E |
| Famotidine | Vitamin B12 |
| Famotidine | Vitamin K |
| Famotidine | Riboflavin |
| Famotidine | Niacin |
| Famotidine | Vitamin D |
| Famotidine | Vitamin B6 |
| Famotidine | Folic acid |
| Famotidine | Pyridoxine |
| Famotidine | Thiamine |
| Famotidine | Pantothenic acid |
| Famotidine | Biotin |
| Nizatidine | Vitamin C |
| Nizatidine | Vitamin A |
| Nizatidine | Vitamin E |
| Nizatidine | Vitamin B12 |
| Nizatidine | Vitamin K |
| Nizatidine | Riboflavin |
| Nizatidine | Niacin |
| Nizatidine | Vitamin D |
| Nizatidine | Vitamin B6 |
| Nizatidine | Folic acid |
| Nizatidine | Pyridoxine |
| Nizatidine | Thiamine |
| Nizatidine | Pantothenic acid |
| Nizatidine | Biotin |
| Ranitidine | Vitamin C |
| Ranitidine | Vitamin A |
| Ranitidine | Vitamin E |
| Ranitidine | Vitamin B12 |
| Ranitidine | Vitamin K |
| Ranitidine | Riboflavin |
| Ranitidine | Niacin |
| Ranitidine | Vitamin D |
| Ranitidine | Vitamin B6 |
| Ranitidine | Folic acid |
| Ranitidine | Pyridoxine |
| Ranitidine | Thiamine |
| Ranitidine | Pantothenic acid |
| Ranitidine | Biotin |

Any of the formulations detailed in Table C may further include a mineral, organic acid, drug, excipient, buffering agent, or any combination of these additional ingredients. By way of non-limiting example, an exemplary formulation may include a proton pump inhibitor, calcium or iron, and a vitamin selected from C, a B vitamin, and vitamin D. Optionally, this formulation may include an organic acid selected from succinic acid, ascorbic acid and glutamic acid.

In an exemplary formulation for the treatment or prevention of calcium malabsorption and in particular, osteoperosis, the pharmaceutical composition may include an organic acid, calcium, vitamin D, and a biphosphonate. This formulation may also include an estrogen or a SERM. Optionally, this formulation may also include a proton pump inhibitor. Specific formulations are described in more detail in the examples.

In an exemplary embodiment for the treatment or prevention of iron malabsorption and in particular, anemia, the pharmaceutical composition may include an organic acid, any of the iron sources detailed herein, and vitamin C. Exemplary organic acids include fumaric acid and succinic acid. Optionally, this formulation may also include a proton pump inhibitor. Specific formulations are described in more detail in the examples.

It is contemplated that, if appropriate, that one or more of the ingredients forming the pharmaceutical composition of the present invention can exist in tautomeric, geometric or stereoisomeric forms without departing from the scope of the invention. The present invention contemplates all such compounds, including cis- and trans-geometric isomers, E- and Z-geometric isomers, R- and S-enantiomers, diastereomers, d-isomers, I-isomers, the racemic mixtures thereof and other mixtures thereof. Pharmaceutically acceptable salts of such tautomeric, geometric or stereoisomeric forms are also included within the invention. The terms “cis” and “trans”, as used herein, denote a form of geometric isomerism in which two carbon atoms connected by a double bond will each have a hydrogen atom on the same side of the double bond (“cis”) or on opposite sides of the double bond (“trans”). Some of the compounds described contain alkenyl groups, and are meant to include both cis and trans or “E” and “Z” geometric forms. Furthermore, some of the compounds described contain one or more stereocenters and are meant to include R, S, and mixtures of R and S forms for each stereocenter present.

Moreover, one or more of the ingredients forming the pharmaceutical composition of the present invention may be in the form of free bases or pharmaceutically acceptable acid addition salts thereof. The term “pharmaceutically-acceptable salts” are salts commonly used to form alkali metal salts and to form addition salts of free acids or free bases. The nature of the salt may vary, provided that it is pharmaceutically acceptable. Suitable pharmaceutically acceptable acid addition salts of compounds for use in the present methods may be prepared from an inorganic acid or from an organic acid. Examples of such inorganic acids are hydrochloric, hydrobromic, hydroiodic, nitric, carbonic, sulfuric and phosphoric acid. Appropriate organic acids may be selected from aliphatic, cycloaliphatic, aromatic, araliphatic, heterocyclic, carboxylic and sulfonic classes of organic acids, examples of which are formic, acetic, propionic, succinic, glycolic, gluconic, lactic, malic, tartaric, citric, ascorbic, glucuronic, maleic, fumaric, pyruvic, aspartic, glutamic, benzoic, anthranilic, mesylic, 4-hydroxybenzoic, phenylacetic, mandelic, embonic (pamoic), methanesulfonic, ethanesulfonic, benzenesulfonic, pantothenic, 2-hydroxyethanesulfonic, toluenesulfonic, sulfanilic, cyclohexylaminosulfonic, stearic, algenic, hydroxybutyric, salicylic, galactaric and galacturonic acid. Suitable pharmaceutically-acceptable base addition salts of compounds of use in the present methods include metallic salts made from aluminum, calcium, lithium, magnesium, potassium, sodium and zinc or organic salts made from N,N′-dibenzylethylenediamine, chloroprocaine, choline, diethanolamine, ethylenediamine, meglumine-(N-methylglucamine) and procaine. All of these salts may be prepared by conventional means from the corresponding compound by reacting, for example, the appropriate acid or base with the one or more of the corresponding compounds set forth herein.

### (II) Pharmaceutical Dosage Forms

The pharmaceutical compositions detailed herein may be manufactured in one or several dosage forms. Suitable dosage forms include a tablet, including a suspension tablet, a chewable tablet, an effervescent tablet or caplet; a pill; a powder such as a sterile packaged powder, a dispensable powder, and an effervescent powder; a capsule including both soft or hard gelatin capsules such as HPMC capsules; a lozenge; a sachet; a sprinkle; a reconstitutable powder or shake; a troche; pellets; granules; liquids; suspensions; emulsions; or semisolids and gels. Alternatively, the pharmaceutical compositions may be incorporated into a food product or powder for mixing with a liquid, or administered orally after only mixing with a non-foodstuff liquid. The pharmaceutical compositions, in addition to being suitable for administration in multiple dosage forms, may also be administered with various dosage regimens, as detailed more precisely below.

The amount and types of ingredients (i.e., pH lowering agents, agents that increase pH, minerals, vitamins, drugs etc), and other excipients useful in each of these dosage forms are described throughout the specification and examples. It should be recognized that where a combination of ingredients and/or excipient, including specific amounts of these components, is described with one dosage form that the same combination could be used for any other suitable dosage form. Moreover, it should be understood that one of skill in the art would, with the teachings found within this application, be able to make any of the dosage forms listed above by combining the amounts and types of ingredients administered as a combination in a single dosage form or a separate dosage forms and administered together as described in the different sections of the specification.

The particle size of the ingredients forming the pharmaceutical composition may be an important factor that can effect bioavailability, blend uniformity, segregation, and flow properties. In general, smaller particle sizes of a drug, such as a proton pump inhibitor, increases the bioabsorption rate of the drug with substantially poor water solubility by increasing the surface area. The particle size of the drug and excipients can also affect the suspension properties of the pharmaceutical formulation. For example, smaller particles are less likely to settle and therefore form better suspensions. In various embodiments, the average particle size of the dry powder of the various ingredients (which can be administered directly, as a powder for suspension, or used in a solid dosage form) is less than about 500 microns in diameter, or less than about 450 microns in diameter, or less than about 400 microns in diameter, or less than about 350 microns in diameter, or less than about 300 microns in diameter, or less than about 250 microns in diameter, or less than about 200 microns in diameter, or less than about 150 microns in diameter, or less than about 100 microns in diameter, or less than about 75 microns in diameter, or less than about 50 microns in diameter, or less than about 25 microns in diameter, or less than about 15 microns in diameter. In some applications the use of particles less than 15 microns in diameter may be advantageous. In these cases colloidal or nanosized particles in the particle size range of 15 microns down to 10 nanometers may be advantageously employed.

The pharmaceutical compositions of the present invention can be manufactured by conventional pharmacological techniques. Conventional pharmacological techniques include, e.g., one or a combination of methods: (1) dry mixing, (2) direct compression, (3) milling, (4) dry or non-aqueous granulation, (5) wet granulation, or (6) fusion. See, e.g., Lachman et al., The Theory and Practice of Industrial Pharmacy (1986). Other methods include, e.g., prilling, spray drying, pan coating, melt granulation, granulation, wurster coating, tangential coating, top spraying, extruding, coacervation and the like.

### (III) Time Controlled Formulations

The pharmaceutical compositions of the invention may be manufactured into one or several dosage forms detailed above and formulated for the controlled, sustained or timed release of one or more of the ingredients. In this context, typically one or more of the ingredients forming the pharmaceutical composition is microencapsulated or dry coated prior to being formulated into one of the above forms. By varying the amount and type of coating and its thickness, the timing and location of release of a given ingredient or several ingredients (in either the same dosage form, such as a multi-layered capsule, or different dosage forms) may be varied.

The coating can and will vary depending upon a variety of factors, including the particular ingredient, and the purpose to be achieved by its encapsulation (e.g., flavor masking, maintenance of structural integrity, or formulation for time release). The coating material may be a biopolymer, a semi-synthetic polymer, or a mixture thereof. The microcapsule may comprise one coating layer or many coating layers, of which the layers may be of the same material or different materials. In one embodiment, the coating material may comprise a polysaccharide or a mixture of saccharides and glycoproteins extracted from a plant, fungus, or microbe. Non-limiting examples include corn starch, wheat starch, potato starch, tapioca starch, cellulose, hemicellulose, dextrans, maltodextrin, cyclodextrins, inulins, pectin, mannans, gum arabic, locust bean gum, mesquite gum, guar gum, gum karaya, gum ghatti, tragacanth gum, funori, carrageenans, agar, alginates, chitosans, or gellan gum. In another embodiment, the coating material may comprise a protein. Suitable proteins include, but are not limited to, gelatin, casein, collagen, whey proteins, soy proteins, rice protein, and corn proteins. In an alternate embodiment, the coating material may comprise a fat or oil, and in particular, a high temperature melting fat or oil. The fat or oil may be hydrogenated or partially hydrogenated, and preferably is derived from a plant. The fat or oil may comprise glycerides, free fatty acids, fatty acid esters, or a mixture thereof. In still another embodiment, the coating material may comprise an edible wax. Edible waxes may be derived from animals, insects, or plants. Non-limiting examples include beeswax, lanolin, bayberry wax, carnauba wax, and rice bran wax. The coating material may also comprise a mixture of biopolymers. As an example, the coating material may comprise a mixture of a polysaccharide and a fat.

In an exemplary embodiment, the coating may be an enteric coating. The enteric coating generally will provide for controlled release of the ingredient, such that drug release can be accomplished at some generally predictable location in the lower intestinal tract below the point at which drug release would occur without the enteric coating. In certain embodiments, multiple enteric coatings may be utilized. Multiple enteric coatings, in certain embodiments, may be selected to release the ingredient or combination of ingredients at various regions in the lower gastrointestinal tract and at various times.

The enteric coating is typically, although not necessarily, a polymeric material that is pH sensitive. A variety of anionic polymers exhibiting a pH-dependent solubility profile may be suitably used as an enteric coating in the practice of the present invention to achieve delivery of the active to the lower gastrointestinal tract. Suitable enteric coating materials include, but are not limited to: cellulosic polymers such as hydroxypropyl cellulose, hydroxyethyl cellulose, hydroxypropyl methyl cellulose, methyl cellulose, ethyl cellulose, cellulose acetate, cellulose acetate phthalate, cellulose acetate trimellitate, hydroxypropylmethyl cellulose phthalate, hydroxypropylmethyl cellulose succinate and carboxymethylcellulose sodium; acrylic acid polymers and copolymers, preferably formed from acrylic acid, methacrylic acid, methyl acrylate, ammonio methylacrylate, ethyl acrylate, methyl methacrylate and/or ethyl methacrylate (e.g., those copolymers sold under the trade name “Eudragit”); vinyl polymers and copolymers such as polyvinyl pyrrolidone, polyvinyl acetate, polyvinylacetate phthalate, vinylacetate crotonic acid copolymer, and ethylene-vinyl acetate copolymers; and shellac (purified lac). Combinations of different coating materials may also be used to coat a single capsule.

The thickness of a microcapsule coating may be an important factor in some instances. For example, the “coating weight,” or relative amount of coating material per dosage form, generally dictates the time interval between oral ingestion and drug release. As such, a coating utilized for time release of the ingredient or combination of ingredients into the gastrointestinal tract is typically applied to a sufficient thickness such that the entire coating does not dissolve in the gastrointestinal fluids at pH below about 5, but does dissolve at pH about 5 and above. The thickness of the coating is generally optimized to achieve release of the ingredient at approximately the desired time and location.

As will be appreciated by a skilled artisan, the encapsulation or coating method can and will vary depending upon the ingredients used to form the pharmaceutical composition and coating, and the desired physical characteristics of the microcapsules themselves. Additionally, more than one encapsulation method may be employed so as to create a multi-layered microcapsule, or the same encapsulation method may be employed sequentially so as to create a multi-layered microcapsule. Suitable methods of microencapsulation may include spray drying, spinning disk encapsulation (also known as rotational suspension separation encapsulation), supercritical fluid encapsulation, air suspension microencapsulation, fluidized bed encapsulation, spray cooling/chilling (including matrix encapsulation), extrusion encapsulation, centrifugal extrusion, coacervation, alginate beads, liposome encapsulation, inclusion encapsulation, colloidosome encapsulation, sol-gel microencapsulation, and other methods of microencapsulation known in the art. Detailed information concerning materials, equipment and processes for preparing coated dosage forms may be found in Pharmaceutical Dosage Forms: Tablets, eds. Lieberman et al. (New York: Marcel Dekker, Inc., 1989), and in Ansel et al., Pharmaceutical Dosage Forms and Drug Delivery Systems, 6^{th} Ed. (Media, Pa.: Williams & Wilkins, 1995).

### (IV) Split Dosing Treatment Regimes

Because the ingredient or combination of ingredients forming the pharmaceutical composition may be manufactured into one or several dosage forms for controlled, sustained, or timed release of the individual ingredients, this provides methods for achieving a split dosing treatment regime. In this context, a “split-dosing regime” means that different ingredients within the same dosage form or different dosage forms release ingredients at substantially different times and locations to substantially achieve the maximum therapeutic efficacy for each ingredient. For example, it is generally known that proton pump inhibitors tend to lose some of their therapeutic efficacy at night (or approximately 12 to 24 hours after their administration to a subject), often allowing the pH of gastric acid to fall below 4. To further optimize absorption of a mineral, vitamin or drug, as such, a proton pump inhibitor may be formulated for immediate release, and the other ingredients may be formulated for extended release. In this manner, the mineral, vitamin, or drug may be released in the gastrointestinal tract at a time when it can be optimally absorbed when the gastrointestinal tract generally has a lower pH (i.e., at a time when the proton pump inhibitor has lost some therapeutic efficacy). Additionally, to further decrease the pH of the gastrointestinal tract, the organic acid may be formulated for extended release.

As such for split dosing treatment regimes, one or more ingredients may be formulated for immediate release and one or more ingredients may be formulated for extended release. In the context of the present invention, ingredients formulated for “immediate release” are generally substantially dissolved in less than about 20 minutes, less than about 15 minutes, less than about 10 minutes, less than about 5 minutes or less than about 1 minute following oral administration to a subject. Alternatively, ingredients formulated for “extended release” are generally substantially dissolved in more than about 20 minutes. For example, the ingredients formulated for extended release typically may be substantially dissolved in greater than about 20 minutes, greater than about 40 minutes, greater than about 60 minutes, greater than about 90 minutes, greater than about 180 minutes, greater than about 3 hours, greater than about 4 hours, greater than about 5 hours, greater than about 6 hours, greater than about 7 hours, greater than about 8 hours, greater than about 9 hours, greater than about 10 hours, greater than about 11 hours, greater than about 12 hours, greater than about 13 hours, greater than about 14 hours, greater than about 15 hours, greater than about 16 hours, greater than about 17 hours, greater than about 18 hours, greater than about 19 hours, greater than about 20 hours, greater than about 21 hours, greater than about 22 hours, greater than about 23 hours, greater than about 24 hours, or up to about 48 hours following oral administration to a subject.

Using immediate and extended release formulations provides a means for a dosing regime that includes the release into the gastrointestinal tract of an ingredient or combination of ingredients from about 30 minutes to about 90 minutes, from about 3 hours to about 9 hours, from about 6 hours to about 12 hours, or from about 8 to about 16 hours after the release of a different ingredient or combination of ingredients into the gastrointestinal tract. The different ingredients or combination of ingredients may be in the same dosage form or in different dosage forms. More over, in addition to release at different times, the ingredient or combination of ingredients may also be formulated for release at different locations within the gastrointestinal tract. In some embodiments, the ingredient or combination of ingredients may be formulated for release into to the small intestine. In an exemplary embodiment, the ingredient or combination of ingredients are formulated for passage through the stomach and release into the proximal small intestine. In other embodiments, the ingredient or combination of ingredients may be formulated for release into to the large intestine.

In an exemplary embodiment, the proton pump inhibitor and/or H2 blocker is formulated for immediate release, and at least one of an organic acid, vitamin, drug, or mineral is formulated for extended release. In another embodiment, the proton pump inhibitor and/or H2 blocker is formulated for immediate release, the organic acid is formulated for extended release, and at least one of a drug, vitamin, or mineral is formulated for extended release.

### (V) Pharmaceutical Kits

It is contemplated that the ingredients forming the various pharmaceutical compositions of the invention may be formulated into the same dosage form or in separate dosage forms and included in a variety of packaging options. In some embodiments, the proton pump inhibitor and/or H2 blocker is in one dosage form and the organic acid, vitamin, mineral, and/or drug are in different dosage forms. The dosage forms may also be bi-daily, weekly, bi-weekly, monthly, or bimonthly dosages of any of the ingredients. Typically, the dosage form will provide a daily dosage.

The different dosage forms may be packaged separately or they may in be included within the same package contained in different cavities, such as in a strip pack or a blister pack. It is envisioned that any of the pharmaceutical formulations described herein may be packaged in a strip pack or blister pack without departing from the scope of the invention. By way of non-limiting example, a blister pack may include a daily dose of a proton pump inhibitor, an organic acid, and at least one of a vitamin, mineral, or drug. In another example, the blister pack may include a daily dose of a proton pump inhibitor, an iron source, vitamin C, and an organic acid. In a further example, the blister pack may include a daily dose of a proton pump inhibitor, a calcium source, vitamin D, an organic acid, and biphosphonate.

(VI) Methods for Improving Absorption of a Nutrient and/or Drug

The pharmaceutical compositions of the invention may be utilized to enhance or improve the gastrointestinal absorption of a nutrient or drug in a subject. The nutrient may be any of the vitamins, minerals, or drugs detailed herein. In an exemplary embodiment, the pharmaceutical compositions provide improved absorption for nutrients and/or drugs that suffer from malabsorption when the gastrointestinal pH, such as the small intestine, is above about 2, 3, or 4.

Moreover, the subject may include a wide range of subjects including animals and humans. The animal may be an agricultural animal. Suitable examples include, but are not limited to, chicken, beef cattle, dairy cattle, swine, sheep, goat, horse, duck, turkey, and goose. The animal may be a companion animal, such as cat, rabbit, rat, hamster, parrot, horse, or dog. The animal may also be an aquatic animal, such as fish or shellfish. Alternatively, the animal may be a game animal or a wild animal. Non-limiting examples of suitable game animals include buffalo, deer, elk, moose, reindeer, caribou, antelope, rabbit, squirrel, beaver, muskrat, opossum, raccoon, armadillo, porcupine, pheasant quail, and snake. In an exemplary embodiment, the subject is a human.

In a particularly preferred embodiment, the subject is a human that has a sustained gastric pH of greater than about 2, greater than about 3, greater than about 4, or greater than about 5. The increased pH may result from natural or iatrogenic causes. The subject may be on a treatment regime that includes taking a proton pump inhibitor or H2 blocker on a daily basis. Alternatively, the subject may have a disorder, such as hypochlorhydria or achlohydria, in which no or lower than normal levels of gastric acid are produced. This disorder may be due to, for example, the aging process, chronic stress, alcohol consumption, a bacterial infection (i.e. H. pylon), autoimmune disease, or atrophic gastritis. The subject may be at risk for developing or may have an indication or disorder resulting from nutrient malabsorption. Furthermore, the subject may be at risk for malnourishment since acid proteases involved in digestion do not function well at elevated pH levels.

The pharmaceutical compositions of the invention may be used independently to promote and/or maintain nutrient or drug absorption or used in combination with one or more other compositions. By way of non-limiting example, the pharmaceutical composition of the invention may be used independently to promote and/or maintain iron absorption, or used in combination with one or more other compositions used in the treatment of one or more diseases having iron deficiency associated therewith. Such diseases or conditions include, for example, gastrointestinal diseases or conditions that cause blood loss such as for example infectious parasites, such as hookworms, regular use of non-steroidal anti-inflammatory drugs, steroids and/or aspirin, peptic ulcer disease, gastritis, colon cancer, polyps, and inflammatory bowel disease, gastrointestinal diseases or conditions that cause decreased absorption of iron such as tropical sprue, celiac disease, autoimmune disease, gastrectomy, gastric bypass, vagotomy, neurological diseases or conditions such as restless leg syndrome, chronic fatigue, cognitive deficiencies and neuron-development deficiencies, physiological conditions such as sports, menses, lactation, pregnancy, and surgery, infectious diseases such as HIV/AIVS and malaria, chronic diseases such as cancer, rheumatoid arthritis, and chronic renal failure and heavy metal poisoning such as lead, mercury, cadmium, and arsenic. A subject having an iron deficiency may have or be at risk for developing anemia. The pharmaceutical composition of the invention may also be used independently to promote and/or maintain calcium absorption, or used in combination with one or more other compositions used in the treatment of one or more diseases having calcium deficiency associated therewith. Conditions that lead to calcium deficiency include chronic kidney disease, vitamin D deficiency, inadequate sunlight exposure, hypoparathyroidism, dietary deficiency, and hyperphosphatemia. A subject with a calcium deficiency for a prolonged time, may have or be at risk for developing depleted bone calcium stores, may develop bones weak and prone to fracture, and may develop osteoporosis.

The following examples are included to demonstrate preferred embodiments of the invention. It should be appreciated by those of skill in the art that the techniques disclosed in the examples that follow represent techniques discovered by the inventors to function well in the practice of the invention. Those of skill in the art should, however, in light of the present disclosure, appreciate that many changes can be made in the specific embodiments that are disclosed and still obtain a like or similar result without departing from the spirit and scope of the invention, therefore all matter set forth or shown in the accompanying drawings is to be interpreted as illustrative and not in a limiting sense.

### EXAMPLES

The following examples illustrate iterations of formulations of the invention.

### Example 1

### Formulation of Vitamin and Esomeprazole Magnesium Tablet

Tablets comprising vitamins and the proton pump inhibitor, esomeprazole, were formulated using current Good Manufacturing Practices (cGMPs). The ingredients are listed in Table 1.

TABLE 1
Ingredients in Vitamin and Esomeprazole Tablet.

| | | | Item | Ingredient | |
|---|---|---|---|---|---|
| Label Claim | | + % | No. | (Source Material) | mg/dose |
| 151.0 | mg | | 1 | Iron - 70.0 mg | 350.0 |
| | | | | (Ferrous Asparto Glycinate) | |
| | | | | (20% Fe/7% Succinic Acid) | |
| | | | 2 | Iron - 81.0 mg | 273.8 |
| | | | | [Ferrous Fumarate 90% (PDI, #94446)] | |
| | | | | (29.58% Fe) | |
| 150.0 | mg | | 3 | Succinic Acid | 125.5 |
| (125.5 | mg) | | | (Succinic Acid, FCC) | |
| (24.5 | mg) | | | (Ferrous Asparto Glycinate, 7% of Succinic Acid) | |
| 200.0 | mg | 10 | 4 | Vitamin C - 140 mg | 158.8 |
| | | | | [Ascorbic Acid (97% Direct Compression) | |
| | | | 5 | Vitamin C - 60.0 mg | 81.5 |
| | | | | [Calcium Ascorbate (Ester-C, | |
| | | | | Pharmaceutical Grade)] | |
| | | | | (81.0% Vitamin C) | |
| 10.0 | mcg | 25 | 6 | Cyanocobalamin | 1.25 |
| | | | | [Cyanocobalamin (1% Spray Dried, B12)] | |
| 1.0 | mg | 20 | 7 | Folic Acid | 1.304 |
| | | | | (Folic Acid, USP) (92% of Folic Acid) | |
| | | | 8 | Lactose, Monohydrate, NF (Modified, #316) | 143.5 |
| | | | 9 | Povidone, USP (K-29/32) | 32.4 |
| | | | 10 | Microcrystalline Cellulose, NF (PH302) | 170.946 |
| | | | 11 | Silicon Dioxide, NF (Syloid 72FP) | 22.5 |
| | | | 12 | Croscarmellose Sodium, NF | 75.0 |
| | | | 13 | Magnesium Stearate, NF | 13.5 |
| | | | 14 | Opadry II White TY-22-7719 | **27.0 |
| | | | 15 | Opadry II red 85G15414 | **61.0 |
| | | | 16 | Water, Purified, USP | * |
| 20.0 | mg | | 17 | Esomeprazole Magnesium (92.5%) | 21.5 |

* Does not appear in finished product.
**This amount includes an overage due to manufacturing losses.

### Example 2

### Formulation of Calcium/Iron with Vitamin D and Esomeprazole Magnesium Tablet

Tablets comprising calcium, iron, vitamin D and the proton pump inhibitor, esomeprazole, were formulated using cGMPs with the ingredients listed in Table 2.

TABLE 2
Ingredients in Calcium/Iron with Vitamin D and Esomeprazole Tablet.

| | | | | Ingredient | |
|---|---|---|---|---|---|
| Label Claim | | + % | Item No. | (Source Material) | mg/dose |
| 20.0 | mg | | 1 | Esomeprazole Magnesium (92.5%) | 21.5 |
| 500.0 | mg | | 2 | Calcium Carbonate, USP (40.0% C) | 1250.0 |
| | | | 3 | Fumaric Acid | 200.0 |
| 400 | IU | 20 | 4 | Vitamin D (Cholecalciferyl 500 MIU/g D₃) | 0.96 |
| | | | 5 | Sodium Lauryl Sulfate, NF | 3.22 |
| | | | 6 | Croscarmellose Sodium, NF | 33.0 |
| | | | 7 | Silicon Dioxide, Colloidal, NF | 3.0 |
| | | | 8 | Hydrogenated Vegetable Oil, NF | 11.2 |
| | | | 9 | Magnesium Stearate, NF | 9.0 |
| | | | 10 | Water, Purified USP | * |
| | | | 11 | Opadry II White TY-22-7719 | **60.0 |
| | | | 12 | Carnauba Wax, NF | 0.060 |

* Does not appear in the finished product.
**This amount includes an overage due to manufacturing losses.

### Example 3

### Formulation of Carvedilol and Omeprazole Tablet

Tablets comprising the non-selective beta blocker, carvedilol, and the proton pump inhibitor, omeprazole, were formulated using cGMPs with the ingredients listed in Table 3.

TABLE 3
Ingredients in Carvedilol and Omeprazole Tablet.

| Label | | Item | Ingredient | |
|---|---|---|---|---|
| Claim | + % | No. | (Source Material) | mg/dose |
| 25.0 mg | | 1 | Carvedilol | 25.0 |
| 20.0 mg | | 2 | Omeprazole (Omeprazole Magnesium) | 20.6 |
| | | 3 | Lactose, Hydrous, USP | 25.0 |
| | | 4 | Silicon Dioxide, Colloidal, NF | 0.5 |
| | | 5 | Microcrystalline Cellulose | 50.0 |
| | | 6 | Succinic Acid | 150.0 |
| | | 7 | Sodium Stearyl Fumarate | 4.1 |
| | | 8 | Croscarmellose Sodium | 12.0 |

What is claimed is:

1. A pharmaceutical composition comprising a first agent that increases the pH of the stomach, a second agent that is a pH lowering agent, and at least one of a third agent selected from the group consisting of a vitamin, mineral, and drug.

2. The pharmaceutical composition of claim 1, wherein the first agent is a proton pump inhibitor.

3. The pharmaceutical composition of claim 2, wherein the proton pump inhibitor is selected from the group consisting of omeprazole, hydroxyomeprazole, esomeprazole, tenatoprazole, lansoprazole, pantoprazole, rabeprazole, dontoprazole, habeprazole, perprazole, ransoprazole, pariprazole, and leminoprazole.

4. The pharmaceutical composition of claim 1, wherein the first agent is a H2 blocker.

5. The pharmaceutical composition of claim 4, wherein the H2 blocker is selected from the group consisting of cimetidine, famotidine, nizatidine, and ranitidine.

6. The pharmaceutical composition of claim 1, wherein the second agent is an organic acid selected from the group consisting of aliphatic, cycloaliphatic, aromatic, heterocyclic, carboxylic and sulfonic classes of organic acids.

7. The pharmaceutical composition of claim 6, wherein the organic acid is selected from the group consisting of formic, acetic, propionic, succinic, glycolic, gluconic, lactic, malic, tartaric, citric, ascorbic, glucuronic, maleic, fumaric, pyruvic, aspartic, glutamic, benzoic, anthranilic, mesylic, stearic, salicylic, p-hydroxybenzoic, phenylacetic, mandelic, embonic (pamoic), methanesulfonic, ethanesulfonic, benzenesulfonic, pantothenic, toluenesulfonic, 2-hydroxyethanesulfonic, sulfanilic, cyclohexylaminosulfonic, algenic, β-hydroxybutyric, galactaric, and galacturonic acid.

8. The pharmaceutical composition of claim 1, wherein the third agent is a vitamin selected from the group consisting of vitamin C, vitamin A, vitamin E, vitamin B12, vitamin K, riboflavin, niacin, vitamin D, vitamin B6, folic acid, pyridoxine, thiamine, pantothenic acid, and biotin.

9. The pharmaceutical composition of claim 1, wherein the third agent is a mineral selected from the group consisting of calcium, chromium, copper, iodine, iron, magnesium, manganese, molybdenum, phosphorus, potassium, selenium, and zinc.

10. The pharmaceutical composition of claim 1, wherein the third agent is a drug selected from the group consisting of acid/alkaline-labile drugs, pH dependent drugs, and drugs that are weak acids or weak bases.

11. The pharmaceutical composition of claim 1, wherein the first agent, second agent, and third agent are formulated into a single dosage form.

12. The pharmaceutical composition of claim 11, wherein the first agent, second agent and third agent are enteric coated, and the second agent and third agent are released substantially simultaneously at approximately the same location in the small intestine.

13. The pharmaceutical composition of claim 12, wherein the first agent is a proton pump inhibitor, the second agent is an organic acid, and the third agent is a vitamin selected from the group consisting of vitamin C, vitamin A, vitamin E, vitamin B12, vitamin K, riboflavin, niacin, vitamin D, vitamin B6, folic acid, pyridoxine, thiamine, pantothenic acid, and biotin.

14. The pharmaceutical composition of claim 12, wherein the first agent is a proton pump inhibitor, the second agent is an organic acid, and the third agent is a mineral selected from the group consisting of calcium, chromium, copper, iodine, iron, magnesium, manganese, molybdenum, phosphorus, potassium, selenium, and zinc.

15. The pharmaceutical composition of claim 12, wherein the first agent is a proton pump inhibitor, the second agent is an organic acid, and the third agent is a drug selected from the group consisting of an antibiotic, antifungal, anti-retroviral, cardiac, and combinations thereof.

16. The pharmaceutical composition of claim 11, wherein the single dosage form is formulated for a time released, split dosing regimen such that the second and third agents are each released substantially simultaneously from about 30 minutes to about 90 minutes after the first agent is released.

17. The pharmaceutical composition of claim 11, wherein the single dosage form is formulated for a time released, split dosing regimen such that the second and third agents are each released substantially simultaneously from about 1 hour to about 4 hours after the first agent is released.

18. The pharmaceutical composition of claim 11, wherein the single dosage form is formulated for a time released, split dosing regimen such that the second and third agents are each released substantially simultaneously from about 3 hours to about 9 hours after the first agent is released.

19. The pharmaceutical composition of claim 11, wherein the single dosage form is formulated for a time released, split dosing regimen such that the second and third agents are each released substantially simultaneously from about 6 hours to about 12 hours after the first agent is released.

20. The pharmaceutical composition of claim 11, wherein the single dosage form is formulated for a time released, split dosing regimen such that the second and third agents are each released substantially simultaneously from about 8 hours to about 16 hours after the first agent is released.

21. The pharmaceutical composition of claim 20, wherein the first agent is a proton pump inhibitor, the second agent is an organic acid, and the third agent is a vitamin selected from the group consisting of vitamin C, vitamin A, vitamin E, vitamin B12, vitamin K, riboflavin, niacin, vitamin D, vitamin B6, folic acid, pyridoxine, thiamine, pantothenic acid, and biotin.

22. The pharmaceutical composition of claim 20, wherein the first agent is a proton pump inhibitor, the second agent is an organic acid, and the third agent is a mineral selected from the group consisting of calcium, chromium, copper, iodine, iron, magnesium, manganese, molybdenum, phosphorus, potassium, selenium, and zinc.

23. The pharmaceutical composition of claim 20, wherein the first agent is a proton pump inhibitor, the second agent is an organic acid, and the third agent is a drug selected from the group consisting of an antibiotic, antifungal, anti-retroviral, cardiac, and combinations thereof.

24. The pharmaceutical composition of claim 20, wherein the first agent, the second agent, and the third agent are enteric coated.

25. The pharmaceutical composition of claim 1, wherein the first agent, second agent, and third agent are formulated into several dosage forms.

26. The pharmaceutical composition of claim 25, wherein first agent is formulated in a dosage form by itself, and the second agent and third agent are together formulated into a single dosage form.

27. The pharmaceutical composition of claim 26, wherein the two dosage forms are packaged together in a blister pack.

28. The pharmaceutical composition of clam 25, wherein the first agent, second agent, and third agent are each formulated into separate dosage forms.

29. The pharmaceutical composition of claim 28, wherein the three dosage forms are packaged together in a blister pack.

30. The pharmaceutical composition of claim 1, further comprising a biphosphonate.

31. The pharmaceutical composition of claim 1, wherein the first agent is esomeprazol, the second agent is succinic acid, and the third agent comprises calcium and vitamin D.

32. The pharmaceutical composition of claim 1, wherein the first agent is esomeprazol, the second agent is succinic acid, and the third agent comprises iron and vitamin D.

33. The pharmaceutical composition of claim 1, wherein the first agent is esomeprazol, the second agent is succinic acid, and the third agent comprises iron, vitamin C, folic acid, and cyanocobalamin.

34. The pharmaceutical composition of claim 1, wherein the first agent is omeprazole, the second agent is succinic acid, and the third agent is digoxin.

35. The pharmaceutical composition of claim 1, further comprising at least one pharmaceutically acceptable excipient.

36. The pharmaceutical composition of claim 1, wherein the first agent is formulated in a dosage form selected from the group consisting of a tablet, a pill, a powder, a capsule, a lozenge, a sachet, a sprinkle, a troche, a pellet, and a liquid; and the second agent is formulated in a dosage form that is different from the first agent and selected from the group consisting of a tablet, a pill, a powder, a capsule, a lozenge, a sachet, a sprinkle, a troche, a pellet, and a liquid.

37. A multi-layered pharmaceutical composition comprising at least one layer having a first agent that increases the pH of the stomach, and at least one layer having at least one of a second agent selected from a mineral, and a vitamin, the second agent comprising an enteric coating.

38. The multi-layered pharmaceutical composition of claim 37, wherein the first agent is a proton pump inhibitor that is enteric coated.

39. A pharmaceutical composition comprising a first agent that increases the pH of the stomach, and a second agent that is a pH-lowering agent, the second agent being enteric coated and released in the small intestine or large intestine.

40. A method for improving the absorption of at least one first agent selected from the group consisting of a nutrient, a vitamin, a mineral, and a drug in a subject, the method comprising co-administering to the subject the first agent and a second agent that is a pH lowering agent.

41. The method of claim 40, wherein the subject has a sustained stomach pH of greater than about 3

42. The method of claim 41, wherein the subject is on a treatment regime that comprises taking either a H2 blocker or a proton pump inhibitor on a daily basis.

43. The method of claim 42, wherein the subject is administered a proton pump inhibitor, the first agent, and the second agent substantially simultaneously.

44. The method of claim 40, wherein first agent and second agent are administered in a formulation such that they are released in the small intestine.

45. The method of claim 40, wherein the second agent is an organic acid.

46. A method for improving the absorption of calcium in a subject, the method comprising co-administering to the subject calcium and an organic acid.

47. The method of claim 46, further comprising administering vitamin D and biphosphonate.

48. The method of claim 46, wherein the subject has a sustained stomach pH of greater than about 3.

49. The method of claim 48, wherein the subject is on a treatment regime that comprises taking either a H2 blocker or a proton pump inhibitor on a daily basis.

50. The method of claim 49, wherein the subject is administered the proton pump inhibitor, the calcium, and the organic acid substantially simultaneously.

51. The method of claim 50, wherein calcium and the organic acid are administered in a formulation such that they are released in the small intestine.

52. The method of 51, further comprising administering vitamin D and biphosphonate.

53. A method for improving the absorption of iron in a subject, the method comprising co-administering to the subject iron and an organic acid.

54. The method of claim 53, further comprising administering vitamin C.

55. The method of claim 53, wherein the subject has a sustained stomach pH of greater than about 3

56. The method of claim 55, wherein the subject is on a treatment regime that comprises taking either a H2 blocker or a proton pump inhibitor on a daily basis.

57. The method of claim 56, wherein the subject is administered the proton pump inhibitor, the iron, and the organic acid substantially simultaneously.

58. The method of claim 56, wherein iron and the organic acid are administered in a formulation such that they are released in the small intestine.

59. The method of 58, further comprising administering vitamin C.
